(19) United States
(12) Patent Application Publication
(10) Pub. No.: US 2019/0105348 A1
(43) Pub. Date: Apr. 11, 2019

(54) CHIMERIC RECEPTORS AND USES THEREOF IN IMMUNE THERAPY

(71) Applicant: Unum Therapeutics Inc., Cambridge, MA (US)

(72) Inventors: Seth Ettenberg, Cambridge, MA (US); Eugene Choi, Cambridge, MA (US)

(73) Assignee: Unum Therapeutics Inc., Cambridge, MA (US)

(21) Appl. No.: 16/091,884

(22) PCT Filed: Apr. 10, 2017
(86) PCT No.: PCT/US17/26752

§ 371 Date: Oct. 5, 2018

Related U.S. Application Data
(60) Provisional application No. 62/320,372, filed on Apr. 8, 2016.

(51) Int. Cl. A61K 35/17; A61K 39/395; C07K 16/28; C07K 16/30; A61P 35/00

(52) U.S. Cl. CPC A61K 35/17; A61K 39/39558; C07K 16/283; C07K 16/30; A61K 2039/505; A61P 35/00; C07K 2317/732; C07K 2317/41; C07K 16/2833

(57) ABSTRACT

Disclosed herein are methods of using immune cells expressing chimeric receptors and bipartite targeting ligands for immunotherapy of cancer and other diseases.

### BACKGROUND OF DISCLOSURE

Cancer immunotherapy, including cell-based therapy, antibody therapy and cytokine therapy, is used to provoke immune responses attacking tumor cells. It is a promising option for treating various types of cancer because of its potential to evade genetic and cellular mechanisms of drug resistance, and to target tumor cells while sparing normal tissues. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., NEnglJ Med. 1979; 300(19):1068-1073; Porter et al., NEnglJ Med. 1994; 330(2):100-106; Kolb et al., Blood. 1995; 86(5):2041-2050; Slavin et al., Blood. 1996; 87(6):2195-2204; and Appelbaum, Nature. 2001; 411(6835):385-389.

Cell-based therapy may involve cytotoxic T cells having reactivity skewed toward cancer cells. Eshhar et al., Proc. Natl. Acad. Sci. U.S.A; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric antigen receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25; 365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF DISCLOSURE

The present disclosure is based on the use of immune cells expressing chimeric receptors combined with a bipartite targeting ligand, which is capable of binding to both the chimeric receptor and a T cell epitope of interest that is complexed with a major histocompatibility complex (MHC) molecule. Such immune cells can target cells presenting the T cell epitope of interest and are effective in immunotherapy for various diseases and disorders, for example, cancer, inflammatory diseases, or infectious diseases.

Accordingly, one aspect of the present disclosure features a method for treating a disease, the method comprising administering to a subject in need thereof
(i) a therapeutically effective amount of an immune cell that expresses a chimeric receptor comprising: (a) an Fc binding domain, and (b) a cytoplasmic signaling domain; and
(ii) a therapeutically effective amount of a bipartite targeting ligand, wherein the bipartite targeting ligand binds the chimeric receptor. The disease to be treated by the methods described herein can be an inflammatory disorder, an infectious disease, or cancer. The immune cell can be administered prior to, concurrently, or after the bipartite targeting ligand.

In some embodiments, the chimeric receptor may further comprise one or more additional domains, for example, a transmembrane domain; a co-stimulatory signaling domain; a hinge domain, or a combination thereof. In one example, the chimeric receptor may comprise, from N-terminus to C-terminus, the Fc binding domain, the transmembrane domain, the co-stimulatory domain, and the cytoplasmic signaling domain. The chimeric receptor may further comprise a hinge domain, which can be located between the Fc binding domain and the transmembrane domain, and/or a signal peptide at the N-terminus.

In some embodiments, the Fc binding domain in any of the chimeric receptors described herein is an extracellular domain of an Fc receptor, which may be a Fcγ receptor (FcγR) (e.g., CD16A, CD16B, CD64A, CD64B, CD64C, CD32A, and CD32B). In one particular example, the FcγR is CD16A. In some examples, the Fc binding domain can be the extracellular domain of a wild-type Fc receptor, when the bipartite targeting ligand for co-use with the chimeric receptor comprises a wild-type Fc fragment. In other examples, the Fc binding domain can be a mutated extracellular domain of an Fc receptor when the bipartite targeting ligand for co-use with the chimeric receptor does not contain a wild-type Fc domain. The Fc binding domain may comprise a mutation at one or more residues involved in the Fc receptor/Fc interaction such that the mutated extracellular domain of the Fc receptor has altered binding activity (enhanced or reduced) to a wild-type Fc fragment. The bipartite targeting ligand can contain an afucosylated Fc domain or a mutated Fc domain comprising a mutation at one or more residues involved in the Fc receptor/Fc interaction such that the bipartite targeting ligand binds the mutated extracellular domain of the Fc receptor in the chimeric receptor.

In some embodiments, the Fc binding domain in the chimeric receptor described herein may be a single-chain antibody that binds to an IgG Fc. For example, the single-chain antibody that binds a wild-type Fc fragment. Alternatively, the single-chain antibody may preferentially bind to a mutated Fc fragment.

In any of the methods described herein, the bipartite targeting ligand can be an antibody that binds a T cell epitope complexed with an MHC molecule. In some embodiments, the bipartite targeting ligand can be an Fc-fusion protein comprising a variable region of a T cell receptor fused to an Fc region of an immunoglobulin G (IgG). Such an Fc-fusion protein may bind to a T cell epitope complexed with a MHC molecule.

In some embodiments, the T cell epitope is a MHC Class I restricted T cell epitope. In other embodiments, the T cell epitope is a MHC Class II restricted T cell epitope. Any of the T cell epitopes described herein may be derived from a cancer antigen (e.g., WT1, HA-1H, NY-ESO-1, and HER-2). The T cell epitopes may also be derived from a viral antigen (e.g., Human Papillomavirus E6, Human Papillomavirus E7, Epstein-Barr Virus LMP2, and HIV gag), or an or an auto-antigen (e.g., a heat-shock protein, such as HSP70). In some examples, the T cell epitope has an amino acid sequence selected from the group consisting of SEQ ID NOs: 75-77, and 91-100. In some instances, the bipartite targeting ligand described herein may bind to a peptide of SEQ ID NO: 75-77 complexed with HLA-A*02:01. In some instance, the bipartite targeting ligand described herein may bind to a peptide of SEQ ID NO: 95 complexed with HLA-B*57. In some instance, the bipartite targeting ligand described herein may bind to a peptide of SEQ ID NO: 96 complexed with HLA-B*2705.

In some embodiments, the T cell epitopes described herein may be derived from a viral antigen.

The subject to be treated by any of the methods described herein can be a human patient having a cancer (e.g., carcinoma, lymphoma, sarcoma, blastoma, and leukemia), an inflammatory disorder (e.g., acute inflammation or chronic inflammation, for example, rheumatoid arthritis, multiple sclerosis, systemic lupus erythematosus), or an infectious disease (e.g., a viral infection such as an infection with Epstein-Barr Virus, Human Papillomavirus, or Human Immunodeficiency Virus).

In any of the methods described herein, the immune cell expressing the chimeric receptor can be a T lymphocyte or an NK cell. In some instances, the T lymphocyte or NK cell is an autologous T lymphocyte or an autologous NK cell isolated from the subject. The autologous T lymphocyte or autologous NK cells can be activated and/or expanded ex vivo, prior to the administration step. In other instances, the T lymphocyte or NK cell can be an allogeneic T lymphocyte or an allogeneic NK cell. The allogeneic T lymphocyte can be engineered to reduce graft-versus-host effects or host-versus-graft effects. For example, the endogenous T cell receptor of the allogeneic T lymphocyte can be inhibited or eliminated. Any of the immune cells described herein may further expresses an exogenous polypeptide comprising a co-stimulatory domain or a ligand of a co-stimulatory factor.

In another aspect, the present disclosure features a kit comprising (a) any of the immune cells described herein, which express the chimeric receptor also described herein, and (b) any of the bipartite targeting ligands described herein.

Also within the scope of the present disclosure are (i) a pharmaceutical composition for use in treating any of the target diseases described herein, the pharmaceutical composition comprising the immune cells described herein, which express the chimeric receptor, the bipartite targeting ligand described herein, or both, and a pharmaceutically acceptable carrier; and (ii) uses of the immune cells and/or the bipartite targeting ligand in manufacturing a medicament for use in treating the target disease.

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 includes a set of graphs demonstrating the binding of afucosylated T1 monoclonal anti-NY-ESO-1/HLA-A2 antibody to target cells (T2 cells) labeled with and without the NY-ESO-1 analog peptide (SLLMWITQV; SEQ ID NO: 101) (FIG. 1, part A) or U266B1 tumor cells expressing endogenous target (FIG. 1, part B).

FIG. 2 includes a series of graphs demonstrating ACTR-T cell activity in the presence or absence of afucosylated T1 monoclonal antibody. Panels A and B: a Jurkat NFAT luciferase reporter assay. Panels C and D: IL-2 production. Panels E and F: proliferation of ACTR and CD3 double positive T-cells in the presence of NY-ESO-1-positive target cells and anti-NY-ESO-1 antibody T1. Target cells tested were T2 cells and were labeled with and without NY-ESO-1 analog peptide (SLLMWITQV; SEQ ID NO: 101) (panels A, C, and E) or U266B1 tumor cells expressing endogenous target (panels B, D, and F).

### DETAILED DESCRIPTION OF DISCLOSURE

Antibody-based immunotherapies are used to treat a wide variety of diseases, including many types of cancer, infectious diseases, and inflammatory disorders. Such a therapy often depends on recognition of cell surface molecules that are differentially expressed on cells for which elimination is desired (e.g., target cells such as cancer cells) relative to normal cells (e.g., non-cancer cells) (Weiner et al. Cell (2012) 148(6): 1081-1084). Several antibody-based immunotherapies have been shown in vitro to facilitate antibody-dependent cell-mediated cytotoxicity of target cells (e.g. cancer cells), and for some it is generally considered that this is the mechanism of action in vivo, as well. ADCC is a cell-mediated innate immune mechanism whereby an effector cell of the immune system, such as natural killer (NK) cells, T cells, monocyte cells, macrophages, or eosinophils, actively lyses target cells (e.g., cancer cells) recognized by specific antibodies.

Described herein are methods and kits for the co-use of immune cells expressing chimeric receptors and bipartite targeting ligands as described herein that are capable of binding to both the chimeric receptor expressed on the immune cells and a specific T cell epitopes, such as those derived from an antigen of interest (e.g., cancer antigens, viral antigens, or autoantigens) that are complexed with the cognate MHC molecules. The bipartite targeting ligands, including certain antibodies and soluble TCR-Fc fusion proteins, can effectively target the immune cell to attack cells expressing the antigen from which the epitope is derived, particularly those present such epitopes via an MHC molecule, or cells presenting the T cell epitope via an MHC molecule. The methods described herein may result in specificity for the effector functions of the immune cell expressing the chimeric receptor for a target cell that presents a peptide from a target antigen in the context of an MHC molecule.

The methods provided herein may enhance ADCC effects against target cells (e.g., cancer cells) that may be recognized by a bipartite targeting ligand. As used herein, a chimeric receptor refers to a non-naturally occurring molecule that can be expressed on the surface of a host cell and comprises an Fc binding domain capable of binding to a bipartite targeting ligand containing an Fc portion, a transmembrane domain, a co-stimulatory domain, and one or more cytoplasmic signaling domains for triggering effector functions of the immune cell expressing the chimeric receptor, wherein at least two domains of the chimeric receptor are derived from different molecules.

Immune cells that express receptors capable of binding such Fc-containing molecules, for example the chimeric receptor molecules described herein, recognize the target cell-bound bipartite targeting ligand and receptor ligand binding stimulates the immune cell to perform effector functions such as release of cytotoxic granules or expression of cell-death-inducing molecules, leading to cell death of the target cell recognized by the bipartite targeting ligands.

### I. Chimeric Receptors (ACTR)

The chimeric receptors described herein comprise an Fc binding domain with binding affinity and specificity for an Fc fragment (“Fc binder”) and a cytoplasmic signaling domain. The chimeric receptor may optionally further comprise a co-stimulatory domain, a transmembrane domain, and/or a hinge domain. The chimeric receptors are configured such that, when expressed on a host cell, the Fc binding domain is located extracellularly for binding to an Fc-containing molecule such as the bipartite targeting ligand described herein and the cytoplasmic signaling domain (as well as the co-stimulatory domain if applicable) is located in the cytoplasm for triggering activation and/or effector signaling. In some embodiments, a chimeric receptor construct as described herein comprises, from N-terminus to C-terminus, the Fc binder, the transmembrane domain, the at least one co-stimulatory signaling domain, and the cytoplasmic signaling domain. In other embodiments, a chimeric receptor construct as described herein comprises, from N-terminus to C-terminus, the Fc binder, the transmembrane domain, the cytoplasmic signaling domains, and the at least one co-stimulatory signaling domain.

Any of the chimeric receptors described herein may further comprise a hinge domain, which may be located at the C-terminus of the Fc binder and the N-terminus of the transmembrane domain. Alternatively or in addition, the chimeric receptor constructs described herein may contain two or more co-stimulatory signaling domains, which may link to each other or be separated by the cytoplasmic signaling domain. The extracellular Fc binder, transmembrane domain, co-stimulatory signaling domain(s), and cytoplasmic signaling domain in a chimeric receptor construct may be linked to each other directly, or via a peptide linker. In some embodiments, the chimeric receptor, which optionally may not comprise a co-stimulatory domain, can be co-expressed in the immune cells with one or more separate polypeptides comprising a co-stimulatory domain or a ligand of a co-stimulation factor, which provide co-stimulatory signals in trans.

A. Fc Binding Domain

The chimeric receptor constructs described herein comprise an Fc binding domain that is an Fc binder, i.e., capable of binding to an Fc fragment (e.g., a wild-type Fc fragment, an afucosylated Fc fragment, or a mutated Fc fragment). A wild-type Fc fragment refers to an Fc fragment having an amino acid sequence of a naturally-occurring Fc fragment and is fucosylated at a glycosylation site therein. A wild-type Fc fragment may be a portion of a molecule produced in a subject endogenously, for example, an endogenous antibody. Alternatively, it may be a portion of a recombinantly produced molecule such as an antibody.

The Fc binder may bind to an Fc portion of any immunoglobulin (e.g., IgG, IgA, IgM, or IgE) of a suitable mammal (e.g., human, mouse, rat, goat, sheep, or monkey) that is present in a bipartite targeting ligand. Any Fc binding domain known in the art may be used for making the chimeric receptors described herein. Suitable Fc binders may be derived from naturally occurring proteins such as mammalian Fc receptors or certain bacterial proteins (e.g., protein A, protein G). Additionally, Fc binders may be synthetic polypeptides, such as single chain antibodies, engineered specifically to bind the Fc fragment of a molecule such as a bipartite targeting ligand with high affinity and specificity. For example, such an Fc binder can be an antibody or an antigen-binding fragment thereof that specifically binds an Fc-containing molecule. Examples include, but are not limited to, a single-chain variable fragment (scFv), a domain antibody, or a nanobody. Alternatively, an Fc binder can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

Any of the Fc binders described herein may have a suitable binding affinity for an Fc fragment, such as the Fc portion of a bipartite targeting ligand described herein. As used herein, “binding affinity” refers to the apparent association constant or K_{A}. The K_{A} is the reciprocal of the dissociation constant, K_{D}. The extracellular ligand-binding domain of an Fc receptor domain of the chimeric receptors described herein may have a binding affinity K_{D} of at least 10⁻⁵, 10⁻⁶, 10⁻⁷, 10⁻⁸, 10, 10⁻¹⁰M or lower for the Fc portion of a bipartite targeting ligand. In some embodiments, the Fc binder has a high binding affinity for an Fc fragment derived from an antibody, isotype of antibodies, or subtype(s) thereof, as compared to the binding affinity of the Fc binder to an Fc fragment derived from another antibody, isotype of antibodies or subtypes thereof. In some embodiments, the extracellular domain of an Fc receptor has specificity for an Fc fragment derived from an antibody, isotype of antibodies, or subtype(s) thereof, as compared to binding of the extracellular domain of an Fc receptor to an Fc fragment derived from another antibody, isotype of antibodies, or subtypes thereof.

The binding affinity or binding specificity for an Fc binding domain or a chimeric receptor comprising an Fc binding domain can be determined by a variety of methods including equilibrium dialysis, equilibrium binding, gel filtration, ELISA, surface plasmon resonance, or spectroscopy.

In some embodiments, the Fc binder is an extracellular domain of a mammalian Fc receptor. As used herein, an “Fc receptor” is a cell surface bound receptor that is expressed on the surface of many immune cells (including B cells, dendritic cells, natural killer (NK) cells, macrophage, neutorphils, mast cells, and eosinophils) and exhibits binding specificity to an Fc domain. Fc receptors are typically comprised of at least 2 immunoglobulin (Ig)-like domains with binding specificity to an Fc (fragment crystallizable) portion of an antibody. In some instances, binding of an Fc receptor to an Fc portion of the bipartite targeting ligand may trigger antibody dependent cell-mediated cytotoxicity (ADCC) effects.

In some embodiments, the Fc binding domain is the extracellular domain of a wild-type Fc receptor, such as those described herein. A wild-type Fc receptor can be any Fc receptor existing in nature, including polymorphism variants found in a natural source.

The Fc receptor used for constructing a chimeric receptor variant as described herein may be a naturally-occurring polymorphism variant (e.g., the CD16 V158 polymorphism variant having the amino acid sequence of SEQ ID NO: 74) and one or more mutations can be introduced at one or more residues involved in the interaction of the polymorphism variant with a naturally-occurring Fc domain. Such mutations may alter (e.g., reduce or enhance) the binding activity of the Fc receptor for a naturally occurring Fc domain.

TABLE 1
Exemplary Polymorphisms in Fc Receptors

| Amino Acid | | | | | | | | | | |
|---|---|---|---|---|---|---|---|---|---|---|
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαεI) | R | S | E | S | Q | S | E | S | I | V |

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcαR1/CD89. Examples of Fc-epsilon receptors include, without limitation, FcεRI and FcεRII/CD23. The table below lists exemplary Fc receptors for use in constructing the chimeric receptors described herein and their binding activity to corresponding Fc domains:

TABLE 2
Exemplary Fc Receptors

| | Principal antibody | |
|---|---|---|
| Receptor name | ligand | Affinity for ligand |
| FcγRI (CD64) | IgG1 and IgG3 | High (Kd~10⁻⁹M) |
| FcγRIIA (CD32) | IgG | Low (Kd > 10⁻⁷M) |
| FcγRIIB1 (CD32) | IgG | Low (Kd > 10⁻⁷M) |
| FcγRIIB2 (CD32) | IgG | Low (Kd > 10⁻⁷M) |
| FcγRIIIA (CD16a) | IgG | Low (Kd > 10⁻⁶M) |
| FcγRIIIB (CD16b) | IgG | Low (Kd > 10⁻⁶M) |
| FcεRI | IgE | High (Kd~10⁻¹⁰M) |
| FcεRII (CD23) | IgE | Low (Kd > 10⁻⁷M) |
| FcαRI (CD89) | IgA | Low (Kd > 10⁻⁶M) |
| Fcα/μR | IgA and IgM | High for IgM, Mid for IgA |
| FcRn | IgG | |

Selection of the ligand binding domain of an Fc receptor for use in the chimeric receptors described herein will be apparent to one of skill in the art. For example, it may depend on factors such as the binding affinity of the Fc receptor to its ligand, an Fc domain, for example of a bipartite targeting ligand for use with the chimeric receptor.

The amino acid sequences of the CD16A V158 polymorphism variant are provided below. SEQ ID NO: 73 represents the amino acid sequence of the precursor receptor (including the signal sequence, which is underlined), and SEQ ID NO: 74 represents the amino acid sequence of the mature protein.

(CD16A V158, precursor protein)
SEQ ID NO: 73
MWQLLLPTALLLLVSAGMRTEDLPKAVVFLEPQWYRVLEKDSVTLKCQ
GAYSPEDNSTQWFHNESLISSQASSYFIDAATVDDSGEYRCQTNLSTL
SDPVQLEVHIGWLLLQAPRWVFKEEDPIHLRCHSWKNTALHKVTYLQN
GKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDW
KDHKFKWRKDPQDK
(CD16A V158, mature protein)
SEQ ID NO: 74
GMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQ
APRWVFKEEDPIHLRCHSWKNTALHKVTYLQNGKGRKYFHHNSDFYIP
KATLKDSGSYFCRGLVGSKNVSSETVNITITQGLAVSTISSFFPPGYQ
VSFCLVMVLLFAVDTGLYFSVKTNIRSSTRDWKDHKFKWRKDPQDK

The extracellular domain of CD16A V158 can be used for constructing the chimeric receptor described herein.

In other embodiments, the Fc binding domain can be a mutated extracellular domain of a mammalian Fc receptor. The mutated Fc receptor extracellular domain may comprise a mutation at one or more residues involved in the Fc receptor/Fc interaction as relative to its wild-type counterpart. In some examples, the mutated Fc receptor extracellular domain is a mutated extracellular domain of a wild-type CD16A, for example, SEQ ID NO: 74.

Any of the mutated Fc binders or variant Fc binding domains described herein may have a suitable binding activity for a modified Fc domain, which may be afucosylated, mutated, or both. Similarly, any of the Fc binders described herein may be subjected to mutation to achieve a suitable (e.g., reduced or eliminated) binding activity to a wild-type Fc fragment. As used here, “binding activity” may encompass the activity induced by interaction of any of the chimeric receptors described herein with a target molecule, such as a desired activity (e.g., ADCC activity, gene expression, etc.). In some embodiments, the binding activity of the mutated Fc binder for a wild-type Fc fragment is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold reduced as compared to binding activity of the Fc binder (in the absence of the one or more mutations) for the wild-type Fc fragment.

The binding activity of a chimeric receptor and/or a chimeric receptor variant comprising a mutated Fc binder and/or its wild-type counterpart for a wild-type Fc fragment can be determined by a variety of methods including physical binding assays, ADCC (cytotoxicity) assays, assessing expression of one or more genes, and/or activation of a signaling pathway in the cell expressing the chimeric receptor and/or a target cell.

In some embodiments, the Fc binders described herein may be subjected to mutation to achieve a suitable (e.g., enhanced, reduced or eliminated) binding affinity to a wild-type Fc fragment. The mutated Fc binding domain of the chimeric receptors described herein may have a binding affinity K_{D} of at least 10⁻⁵, 10⁻⁶, 10⁻⁷, 10⁻⁸, 10⁻⁹, 10⁻¹⁰ M or lower for a wild-type Fc fragment. In some embodiments, the mutated Fc binder has a reduced binding affinity for a specific wild-type Fc fragment, isotype, or subtype(s) thereof, as compared to the binding affinity of the mutated Fc binder to another Fc fragment, isotype of antibodies or subtypes thereof (e.g., an afucosylated antibody or an antibody that comprises one or more mutations relative to a wild-type antibody). In some embodiments, the binding affinity of the mutated Fc binder for a wild-type Fc fragment is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold reduced as compared to binding affinity of the Fc binder (in the absence of the one or more mutations) for the wild-type Fc fragment.

In some embodiments, the mutated Fc binder has an enhanced binding affinity for a specific wild-type Fc fragment, isotype, or subtype(s) thereof, for example, endogenous antibodies, as compared to the binding affinity of the mutated Fc binder to another Fc fragment, isotype of antibodies or subtypes thereof. In some embodiments, the binding affinity of the mutated Fc binder for a wild-type Fc fragment is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold enhanced as compared to binding affinity of the Fc binder (in the absence of the one or more mutations) for the wild-type Fc fragment. Chimeric receptors containing such mutated Fc binders may have an enhanced activity induced by molecules (e.g., endogenous antibodies) containing a wild-type Fc fragment, for example, the effector function of a host cell expressing the chimeric receptor variant, such as enhanced ADCC.

The binding affinity of a chimeric receptor variant comprising a mutated Fc binder or its wild-type counterpart for an Fc domain can be determined by a variety of methods including, without limitation, equilibrium dialysis, equilibrium binding, flow cytometery, gel filtration, ELISA, surface plasmon resonance, or spectroscopy.

In general, the terms “about” and “approximately” mean within an acceptable error range for the particular value as determined by one of ordinary skill in the art, which will depend in part on how the value is measured or determined, i.e., the limitations of the measurement system. For example, in regard to the binding activity of a chimeric receptor variant “about” can mean within an acceptable standard deviation, per the practice in the art. Alternatively, “about” can mean a range of up to ±30%, preferably up to ±20%, more preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The mutated Fc receptor extracellular domain has an altered (e.g., enhanced or reduced) binding activity to a wild-type Fc fragment as described herein but is capable of binding to a modified Fc domain, for example, an afucosylated Fc domain or a mutated Fc domain as described herein, which can be a portion of the bipartite targeting ligand described herein.

Chimeric receptors (antibody-coupled T cell receptors or ACTRs) comprising a mutated Fc receptor extracellular domain would have no or low binding activity to molecules having a wild-type Fc fragment, such as endogenous antibodies. Reducing or eliminating binding of an ACTR variant to the wild-type Fc fragment may result in a reduction of the activity of the ACTR variant induced by molecules (e.g., endogenous antibodies) containing a wild-type Fc fragment, for example, the effector function of a host cell expressing the chimeric receptor variant, such as ADCC. In some embodiments, the Fc binding domain of an ACTR described herein comprises an amino acid sequence that is at least 90% (e.g., 91, 92, 93, 94, 95, 96, 97, 98, 99%) identical to the amino acid sequence of the Fc binding domain of a naturally-occurring Fc-gamma receptor, an Fc-alpha receptor, or an Fc-epsilon receptor. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some examples, the Fc receptor can be CD16A, CD16B, CD32A, CD32B, CD32C, CD64A, CD64B, CD64C, or a variant thereof as described herein. The extracellular ligand-binding domain of an Fc receptor may comprise up to 10 amino acid residue variations (e.g., 1, 2, 3, 4, 5, or 8) relative to the amino acid sequence of the extracellular ligand-binding domain of CD16A, CD16B, CD32A, CD32B, CD32C, CD64A, CD64B, CD64C as described herein. Such Fc domains comprising one or more amino acid variations may be referred to as a variant. Mutation of amino acid residues of the extracellular ligand-binding domain of an Fc receptor may result in an increase in binding affinity for the Fc receptor domain to bind to an antibody, isotype of antibodies, or subtype(s) thereof relative to Fc receptor domains that do not comprise the mutation.

In some embodiments, the one or more mutations in an extracellular domain of an Fc receptor for use in constructing the chimeric receptor described herein may be at residues of the extracellular domain of an Fc receptor that are involved in interaction, directly or indirectly, with a wild-type Fc fragment. Such residues may be identified, for example, by assessing protein models of the interaction between an Fc receptor and an antibody or Fc domain. (See, for example, Lu et al. Proc. Natl. Acad. Sci. USA (2015) 112(3): 833-838; Radaev et al. J. Biol. Chem. (2011) 276 (19) 16469-16477; Mizushima et al. Genes Cells (2011) 11:1071-1080; Ahmed et al. J. Struct. Biol. (2016) 194:78-89; Ferrera et al. Proc. Natl. Acad. Sci. USA (2011) 108(31):12669-12674; and Sondermann et al. Nature (2000) 406 (6793):267-273). In some embodiments, one or more residues of the extracellular domain of an Fc receptor involved in direct interaction, or predicted to be in direct interaction, with a wild-type Fc fragment may be mutated, for example to alter (e.g., enhance or reduce) the direct interaction. In some embodiments, one or more residues of the extracellular domain of an Fc receptor involved in indirect interaction, or predicted to indirectly interact, with a wild-type Fc fragment may be mutated, for example to alter (e.g., enhance or reduce) interaction between the Fc receptor and the wild-type Fc fragment.

As used herein, the term “mutation” may include a substitution mutation in which an amino acid is replaced with a different amino acid, or deletion mutation in which the amino acid at a given position is removed. The binding activity of a mutated Fc binder thus prepared to a wild-type Fc fragment and/or bipartite targeting ligand for use with the chimeric receptor can be verified by conventional methods and/or those described herein.

As would be appreciated by one of skill in the art, Fc receptors belonging to different superfamilies may share similar structure-functional correlation even if their primary amino acid sequences are different. Structural and sequence comparisons among Fc receptors were known in the art. See, e.g., Lu et al. J. Biol. Chem. (2011) 286(47): 40608-40613. Mutation of an amino acid in a corresponding position in an Fc receptor belonging to different families or superfamilies may be made by comparing the secondary and/or tertiary structure of the Fc receptors to identify the relevant functional domains. In some embodiments, residues involved in the interaction between an Fc receptor and an Fc fragment may be identified based on sequence and/or structural alignment with other Fc receptors for which such residues are known, e.g., the FcγR reported in Lu et al., 2011. In some embodiments, the one or more mutations are of residues of the Fc receptor that are located or predicted to be located at the interface between the Fc receptor and an Fc region. In some embodiments, the one or more mutations are located in the Fc fragment binding pocket of the Fc receptor. In some embodiments, the one or more mutations are located in the D2 region of the extracellular domain of an Fc receptor. In some embodiments, the one or more mutations are located outside of the D2 region of the extracellular domain of an Fc receptor.

Without wishing to be bound by any particular theory, the one or more mutations may alter (enhance, reduce, or eliminate) glycosylation of the Fc binding domain, which may thereby reduce binding activity of the mutated Fc receptor to a wild-type Fc fragment. In some embodiments, the immune cells expressing the chimeric receptor are expanded under growth conditions that alter (enhance, reduce, or eliminate) glycosylation of the Fc binding domain portion in the chimeric receptor. In some embodiments, the immune cells expressing the chimeric receptor are modified, for example, to express one or more glycosylation enzymes or glycosylation pathways, resulting in altered (enhanced, reduced, or eliminated) glycosylation of the Fc binding domain portion in the chimeric receptor. In some embodiments, the mutated Fc binder is derived from CD16A. In some embodiments, the CD16A is a natural polymorphism, such as V158, described herein (SEQ ID NO: 74) or F158. It is appreciated in the art that the V158 (or F158) polymorphism is referred to as such and corresponds to the amino acid at position 160 of the CD16A mature protein sequence. In some embodiments, the mutation is a substitution mutation of one or more amino acids corresponding to W92, K122, Y134, H136, V160, F160, G161, K163, and/or N164 in SEQ ID NO: 74 (in boldface above). As used herein, a position in any given sequence that corresponds to a position in a reference sequence refers to the counterpart position in the given sequence relative to the position in the reference sequence, even though the position may be numbered differently in the two sequences (e.g., due to a different numbering system or a different starting position used). Such a counterpart position can be readily identified by aligning the given sequence with the reference sequence following routine practice.

It would also be evident to one of skill in the art that the amino acids corresponding to W92, K122, Y134, H136, G161, K163, and/or N164 of the CD16A mature protein sequence may also be referred to in the art as W90, K120, Y132, H134, G159, K161, and/or N162. Selection of a suitable amino acid to substitute at a particular position will be evident to one of skill in the art and may be based on factors such as the properties of the side chain of the specific amino acid. In some embodiments, the one or more mutations is W92F, W92K, W92R, W92V, K122D, K122E, K122R, K122M, K122L, K122N, Y134W, Y134A, H136Y, H136W, H136F, V160W, V160K, V160D, V160Q, V160N, G161W, G161F, K163D, K163E, N164A and/or N164Q. It should be appreciated that the extracellular ligand-binding domain of an Fc receptor may further comprise mutation of any one or more additional residues that are not involved in the interaction of the Fc receptor and a wild-type Fc fragment. In some embodiments, the just-noted one or more mutations are the only mutations in a mutated Fc binder.

It would be evident to one of skill in the art that similar mutations may be made extracellular ligand-binding domains of different Fc receptors. For example, the corresponding amino acids of extracellular ligand-binding domains of a different Fc receptor may be identified by aligning the amino acid sequence of SEQ ID NO: 74 with the amino acid sequence of the extracellular ligand-binding domains of the different Fc receptor, using sequence alignment algorithms, such as CLUSTALW.

In other embodiments, the mutated Fc receptor can be derived from a non-CD16 receptor, such as CD32 or CD64, or others disclosed herein. The mutation(s) may occur in the residues that are involved in, or predicted to be involved in, direct or indirect interaction with the corresponding a Fc fragment. In some embodiments, the mutation(s) may occur in a domain of the Fc receptor (e.g., the D2 domain) that is involved in, or predicted to be involved in, direct or indirect interaction with an Fc fragment. Such functional domains are either known in the art (see Lu et al., 2011) or can be identified by performing sequence/structural alignment with Fc receptors having known sequence/structure correlation (e.g., FcγR disclosed in Lu et al., 2011). In some examples, the mutated Fc receptor may contain one or more mutations at positions corresponding to W92, K122, Y134, H136, V160, G161, K163, and/or N164 in SEQ ID NO: 74, which can be identified by performing structure/sequence alignment between SEQ ID NO:74 and the parent Fc receptor of the mutated Fc binder.

Also within the scope of the present disclosure are combinations of mutations in the extracellular ligand-binding domain of an Fc receptor. In some embodiments, the mutated extracellular domain of a Fc receptor comprises a mutation of at least 1, 2, 3, 4, 5, 6, 7, 8, 9, or at least 10 residues, relative to the wild-type counterpart, that are involved in interaction between the Fc receptor and a wild-type Fc fragment.

Specific examples of CD16A mutants include CD16A mutant V160Q, V160W, Y134A, K122L, and Y134A/N164Q (mutation positions correspond to positions 160, 134, 122, and 164 in SEQ ID NO: 74). In these specific examples, the called out amino acid substitutions are the only mutations relative to the wild-type CD16A counterpart (e.g., SEQ ID NO:74).

In other embodiments, the Fc binder is derived from a naturally occurring bacterial protein that is capable of binding to the Fc portion of an IgG molecule. A Fc binder for use in constructing a chimeric receptor as described herein can be a full-length protein or a functional fragment thereof. Protein A is a 42 kDa surface protein originally found in the cell wall of the bacterium Staphylococcus aureus. It is composed of five domains that each fold into a three-helix bundle and are able to bind IgG through interactions with the Fc region of most antibodies as well as the Fab region of human VH3 family antibodies. Protein G is an approximately 60-kDa protein expressed in group C and G Streptococcal bacteria that binds to both the Fab and Fc region of mammalian IgGs. While native protein G also binds albumin, recombinant variants have been engineered that eliminate albumin binding.

Such Fc binders for use in constructing the chimeric receptors (ACTRs) described herein may also be created de novo using combinatorial biology or directed evolution methods. Starting with a protein scaffold (e.g., an scFv derived from IgG, a Kunitz domain derived from a Kunitz-type protease inhibitor, an ankyrin repeat, the Z domain from protein A, a lipocalin, a fibronectin type III domain, an SH3 domain from Fyn, or others), amino acid side chains for a set of residues on the surface may be randomly substituted in order to create a large library of variant scaffolds. From large libraries it is possible to isolate rare variants with affinity for a target like the Fc domain by first selecting for binding, followed by amplification by phage, ribosome or cell display. Repeated rounds of selection and amplification can be used to isolate those proteins with the highest affinity for the target.

In some embodiments, the extracellular domain of the chimeric receptor variant described herein may be a single chain antibody fragment that preferentially binds to a mutated Fc fragment as relative to a wild-type Fc fragment. A molecule is said to exhibit “preferential binding” if it reacts or associates more frequently, more rapidly, with greater duration and/or with greater affinity with a particular target antigen than it does with alternative targets. An antibody “preferentially binds” to a target antigen if it binds with greater affinity, avidity, more readily, and/or with greater duration than it binds to other substances. For example, an antibody that preferentially binds to a mutated Fc fragment is an antibody that binds this target antigen with greater affinity, avidity, more readily, and/or with greater duration than it binds to other antigens such as a wild-type Fc fragment. It is also understood by reading this definition that, for example, an antibody that preferentially binds to a first target antigen may or may not specifically or preferentially bind to a second target antigen. As such, “preferential binding” does not necessarily require (although it can include) exclusive binding.

The mutated Fc fragment may contain mutations at a suitable number of positions such that the mutated Fc fragment could induce antibodies having no or low cross reactivity to a wild-type Fc fragment. In some examples, the mutated Fc fragment shares at least 85% sequence identity (e.g., 90%, 95%, or 98%) with a wild-type Fc fragment. In some embodiments, the scFv fragment in the chimeric receptor variant does not bind a wild-type Fc fragment.

B. Transmembrane Domain

In some embodiments, the chimeric receptors described herein further comprise a transmembrane domain. The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor described herein is derived from a Type I single-pass membrane protein. Single-pass membrane proteins include, but are not limited to, CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8α. In some examples, the transmembrane domain is of 4-1BB/CD137. In other examples, the transmembrane domain is of CD28 or CD34. In yet other examples, the transmembrane domain is not derived from human CD8α. In some embodiments, the transmembrane domain of the chimeric receptor is a single-pass alpha helix.

Transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor described herein.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5 or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

C. Co-Stimulatory Signaling Domains

Many immune cells require co-stimulation, in addition to stimulation of an antigen-specific signal, to promote cell proliferation, differentiation and survival, as well as to activate effector functions of the cell. The chimeric receptors described herein comprise at least one co-stimulatory signaling domain. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptors described herein. The type(s) of co-stimulatory signaling domain is selected based on factors such as the type of the immune cells in which the chimeric receptors would be expressed (e.g., T cells, NK cells, macrophages, neutrophils, or eosinophils) and the desired immune effector function (e.g., ADCC effect). Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300α/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof. In other embodiments, the co-stimulatory signaling domain is not derived from 4-1BB.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant. In some embodiments, the chimeric receptors may comprise more than one co-stimulatory signaling domain (e.g., 2, 3 or more). In some embodiments, the chimeric receptor comprises two or more of the same co-stimulatory signaling domains, for example, two copies of the co-stimulatory signaling domain of CD28. In some embodiments, the chimeric receptor comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptors (e.g., immune cells such as T cells, NK cells, macrophages, neutrophils, or eosinophils) and the desired immune effector function. In some embodiments, the chimeric receptor comprises two co-stimulatory signaling domains. In some embodiments, the two co-stimulatory signaling domains are CD28 and 4-1BB. In some embodiments, the two co-stimulatory signaling domains are CD28_{LL→GG} variant and 4-1BB.

Any of the co-stimulatory domains, or a combination thereof, may be part of the chimeric receptors described herein. Chimeric receptors containing a co-stimulatory signaling domain may be co-used (co-introduced into a host cell) with a separate polypeptide, which can be a co-stimulatory factor or comprises the co-stimulatory domain thereof. The separate polypeptide may comprise the same co-stimulatory domain as the chimeric receptor, or a different co-stimulatory domain. Chimeric receptors containing a co-stimulatory signaling domain may also be co-used with a separate polypeptide comprising a ligand of a co-stimulatory factor, which can be the same as or different from that used in the chimeric receptor. See, e.g., Zhao, et al. Cancer Cell (2015) 28:415-428.

Alternatively, chimeric receptors that do not contain a co-stimulatory domain can be co-used (co-introduced into a host cell) with a separate polypeptide, which can be a co-stimulatory factor or comprises the co-stimulatory domain thereof. Chimeric receptors that do not contain a co-stimulatory signaling domain may also be co-used with a separate polypeptide comprising a ligand of a co-stimulatory factor.

D. Cytoplasmic Signaling Domain

Any cytoplasmic signaling domain can be used to construct the chimeric receptors described herein. In general, a cytoplasmic signaling domain relays a signal, such as interaction of an extracellular ligand-binding domain with its ligand, to stimulate a cellular response, such as inducing an effector function of the cell (e.g., ADCC).

In some embodiments, cytoplasmic signaling domain comprises an immunoreceptor tyrosine-based inhibition motif (ITIM). In some embodiments, the cytoplasmic signaling domain comprises an immunoreceptor tyrosine-based activation motif (ITAM). An “ITIM” and an “ITAM,” as used herein, are conserved protein motifs that are generally present in the tail portion of signaling molecules expressed in many immune cells.

The ITIM motif comprises the amino acid sequence S/I/V/LxYxxI/V/L. Upon stimulation of an ITIM, the motif becomes phosphorylated and reduce activation of molecules involved in cell signaling, thereby transducing an inhibitory signal. In some examples, the cytoplasmic domain comprising an ITIM is of a Killer-cell immunoglobulin-like receptor (KIR).

The ITAM motif may comprise two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcεR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same chimeric receptor construct is derived from CD16A.

E. Hinge Domain

In some embodiments, the chimeric receptors described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the chimeric receptor can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO: 82), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more. In some embodiments, the hinge domain is (Gly₄Ser)₃ (SEQ ID NO: 83). In some embodiments, the hinge domain is (Gly₄Ser)₆ (SEQ ID NO: 84). In some embodiments, the hinge domain is (Gly₄Ser)₉ (SEQ ID NO: 85). In some embodiments, the hinge domain is (Gly₄Ser)₁₂ (SEQ ID NO: 86). In some embodiments, the hinge domain is (Gly₄Ser)₁₅ (SEQ ID NO: 87). In some embodiments, the hinge domain is (Gly₄Ser)₃₀ (SEQ ID NO: 88). In some embodiments, the hinge domain is (Gly₄Ser)₄₅ (SEQ ID NO: 89). In some embodiments, the hinge domain is (Gly₄Ser)₆₀ (SEQ ID NO: 90).

In other embodiments, the hinge domain is an extended recombinant polypeptide (XTEN), which is an unstructured polypeptide consisting of hydrophilic residues of varying lengths (e.g., 10-200 amino acid residues, 20-150 amino acid residues, 30-100 amino acid residues, or 40-80 amino acid residues). Amino acid sequences of XTEN peptides will be evident to one of skill in the art and can be found, for example, in U.S. Pat. No. 8,673,860, which is herein incorporated by reference. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

F. Signal Peptide

In some embodiments, the chimeric receptor also comprises a signal peptide (also known as a signal sequence) at the N-terminus of the polypeptide. In general, signal sequences are peptide sequences that target a polypeptide to the desired site in a cell. In some embodiments, the signal sequence targets the chimeric receptor to the secretory pathway of the cell and will allow for integration and anchoring of the chimeric receptor into the lipid bilayer. Signal sequences including signal sequences of naturally occurring proteins or synthetic, non-naturally occurring signal sequences, that are compatible for use in the chimeric receptors described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16. An example signal sequence is provided by amino acid residues 1-16 of SEQ ID NO: 73.

Tables 3-5 provide exemplary chimeric receptors described herein. These exemplary constructs have, from N-terminus to C-terminus in order, the signal sequence, the Fc binder (e.g., an extracellular domain of an Fc receptor), the hinge domain, and the transmembrane, while the positions of the co-stimulatory domain and the cytoplasmic signaling domain can be switched. As one non-limiting example, the chimeric receptor may have a CD8α signal sequence; a CD16A-V158 extracellular domain; no hinge domain; a CD8α TM domain; a 4-1BB co-stimulatory domain; and a CD3ζ cytoplasmic signaling domain. As another non-limiting example, the chimeric receptor may have a CD8a signal sequence; a CD16A-V158 extracellular domain; a CD28 hinge domain; a CD28 TM domain; a CD28 co-stimulatory domain; and a CD3ζ cytoplasmic signaling domain.

TABLE 3
Exemplary chimeric receptors

| Exemplary | | | | | | |
|---|---|---|---|---|---|---|
| AA | | | | | | |
| Sequence | | Extracellular | | | Co- | Cytoplasmic |
| (SEQ ID | Signal | domain of | Hinge | Transmembrane | stimulatory | Signaling |
| NO) | Sequence | Fc receptor | domain | domain | domain | domain |
| 1 | CD8α | CD16A- | CD8α | CD8α | 4-1BB | CD3ζ |
| | | V158 | | | (CD137) | |
| 2 | CD8α | CD16A- | CD8α | 4-1BB (CD137) | 4-1BB | CD3ζ |
| | | V158 | | | (CD137) | |
| 3 | CD8α | CD16A- | CD8α | CD28 | 4-1BB | CD3ζ |
| | | V158 | | | (CD137) | |
| 4 | CD8α | CD16A- | CD8α | CD34 | 4-1BB | CD3ζ |
| | | V158 | | | (CD137) | |
| 5 | CD8α | CD16A- | CD8α | Designed | 4-1BB | CD3ζ |
| | | V158 | | hydrophobic TM | (CD137) | |
| | | | | domain | | |
| 6 | CD8α | CD32A | CD8α | CD8α | 4-1BB | CD3ζ |
| | | | | | (CD137) | |
| 7 | CD8α | CD16A- | CD8α | CD8α | CD28 | CD3ζ |
| | | V158 | | | | |
| 8 | CD8α | CD16A- | CD8α | CD8α | OX40 | CD3ζ |
| | | V158 | | | (CD134) | |
| 9 | CD8α | CD16A- | CD8α | CD8α | CD28 + | CD3ζ |
| | | V158 | | | 4-1BB | |
| 10 | CD8α | CD16A- | None | CD8α | 4-1BB | CD3ζ |
| | | V158 | | | (CD137) | |
| 11 | CD8α | CD16A- | XTEN | CD8α | 4-1BB | CD3ζ |
| | | V158 | | | (CD137) | |

TABLE 4
Exemplary chimeric receptors

| Exemplary | | | | | | |
|---|---|---|---|---|---|---|
| AA | | | | | | |
| Sequence | | Extracellular | | | Co- | Cytoplasmic |
| (SEQ ID | Signal | domain of | Hinge | Transmembrane | stimulatory | Signaling |
| NO) | Sequence | Fc receptor | domain | domain | domain | domain |
| 12 | CD8α | CD16A- | CD8α | CD8α | CD28 LL | CD3ζ |
| | | V158 | | | to GG | |
| | | | | | mutant | |
| 13 | CD8α | CD16A- | CD8α | CD8α | CD28 LL | CD3ζ |
| | | V158 | | | to GG | |
| | | | | | mutant + | |
| | | | | | 4-1BB | |
| 14 | CD8α | CD16A- | CD8α | CD4 | 4-1BB | CD3ζ |
| | | V158 | | | (CD137) | |
| 15 | CD8α | CD16A- | CD8α | CD4 | CD28 LL | CD3ζ |
| | | V158 | | | to GG | |
| | | | | | mutant + | |
| | | | | | 4-1BB | |
| 16 | CD8α | CD16A- | CD8α | FcεRIγ | 4-1BB | CD3ζ |
| | | V158 | | | (CD137) | |
| 17 | CD8α | CD16A- | CD8α | Designed | 4-1BB | CD3ζ |
| | | V158 | | hydrophobic TM | (CD137) | |
| | | | | domain, predicted | | |
| | | | | dimerization | | |

TABLE 5
Exemplary chimeric receptors

| Exemplary | | | | | | |
|---|---|---|---|---|---|---|
| AA | | | | | | |
| Sequence | | Extracellular | | | Co- | |
| (SEQ ID | Signal | domain of | Hinge | Transmembrane | stimulatory | Signaling |
| NO) | Sequence | Fc receptor | domain | domain | domain | domain |
| 18 | CD8α | CD16A- | CD8α | CD8β | 4-1BB | CD3ζ |
| | | V158 | | | (CD137) | |
| 19 | CD8α | CD16A- | CD8α | C16α | 4-1BB | CD3ζ |
| | | V158 | | | (CD137) | |
| 20 | CD8α | CD16A- | CD8α | OX40 (CD134) | 4-1BB | CD3ζ |
| | | V158 | | | (CD137) | |
| 21 | CD8α | CD16A- | CD8α | CD3ζ | 4-1BB | CD3ζ |
| | | V158 | | | (CD137) | |
| 22 | CD8α | CD16A- | CD8α | CD3ε | 4-1BB | CD3ζ |
| | | V158 | | | (CD137) | |
| 23 | CD8α | CD16A- | CD8α | CD3γ | 4-1BB | CD3ζ |
| | | V158 | | | (CD137) | |
| 24 | CD8α | CD16A- | CD8α | CD3δ | 4-1BB | CD3ζ |
| | | V158 | | | (CD137) | |
| 25 | CD8α | CD16A- | CD8α | TCR-α | 4-1BB | CD3ζ |
| | | V158 | | | (CD137) | |
| 26 | CD8α | CD16A- | CD8α | CD32 | 4-1BB | CD3ζ |
| | | V158 | | | (CD137) | |
| 27 | CD8α | CD16A- | CD8α | CD64 | 4-1BB | CD3ζ |
| | | V158 | | | (CD137) | |
| 28 | CD8α | CD16A- | CD8α | VEGFR2 | 4-1BB | CD3ζ |
| | | V158 | | | (CD137) | |
| 29 | CD8α | CD16A- | CD8α | FAS | 4-1BB | CD3ζ |
| | | V158 | | | (CD137) | |
| 30 | CD8α | CD16A- | CD8α | FGFR2B | 4-1BB | CD3ζ |
| | | V158 | | | (CD137) | |
| 31 | CD8α | CD16A- | CD8α | CD8α | 4-1BB | CD3ζ |
| | | F158 | | | (CD137) | |
| 32 | CD8α | CD64A | CD8α | CD8α | 4-1BB | CD3ζ |
| | | | | | (CD137) | |
| 33 | CD8α | CD16A- | IgG1 (hinge- | CD8α | 4-1BB | CD3ζ |
| | | V158 | CH2-CH3) | | (CD137) | |
| 34 | CD8α | CD16A- | IgG1 (hinge- | CD8α | 4-1BB | CD3ζ |
| | | V158 | CH3) | | (CD137) | |
| 35 | CD8α | CD16A- | IgG1 (hinge) | CD8α | 4-1BB | CD3ζ |
| | | V158 | | | (CD137) | |
| 36 | CD8α | CD16A- | CD8-alpha | CD8α | 4-1BB | CD3ζ |
| | | V158 | fragment 1 | | (CD137) | |
| | | | (30 amino | | | |
| | | | acids) | | | |
| 37 | CD8α | CD16A- | CD8-alpha | CD8α | 4-1BB | CD3ζ |
| | | V158 | fragment 2 | | (CD137) | |
| | | | (15 amino | | | |
| | | | acids) | | | |
| 38 | CD8α | CD16A- | (Gly4Ser)x3 | CD8α | 4-1BB | CD3ζ |
| | | V158 | (60 amino | | (CD137) | |
| | | | acids) | | | |
| 39 | CD8α | CD16A- | (Gly4Ser)x6 | CD8α | 4-1BB | CD3ζ |
| | | V158 | (45 amino | | (CD137) | |
| | | | acids) | | | |
| 40 | CD8α | CD16A- | (Gly4Ser)x9 | CD8α | 4-1BB | CD3ζ |
| | | V158 | (30 amino | | (CD137) | |
| | | | acids) | | | |
| 41 | CD8α | CD16A- | (Gly4Ser)x12 | CD8α | 4-1BB | CD3ζ |
| | | V158 | (15 amino | | (CD137) | |
| | | | acids) | | | |
| 42 | CD8α | CD16A- | XTEN (60 | CD8α | 4-1BB | CD3ζ |
| | | V158 | amino acids) | | (CD137) | |
| 43 | CD8α | CD16A- | XTEN (30 | CD8α | 4-1BB | CD3ζ |
| | | V158 | amino acids) | | (CD137) | |
| 44 | CD8α | CD16A- | XTEN (15 | CD8α | 4-1BB | CD3ζ |
| | | V158 | amino acids) | | (CD137) | |
| 45 | CD28 | CD16A- | CD8α | CD8α | 4-1BB | CD3ζ |
| | | V158 | | | (CD137) | |
| 46 | Murine | CD16A- | CD8α | CD8α | 4-1BB | CD3ζ |
| | kappa | V158 | | | (CD137) | |
| | chain | | | | | |
| 47 | CD16 | CD16A- | CD8α | CD8α | 4-1BB | CD3ζ |
| | | V158 | | | (CD137) | |
| 48 | CD8α | CD16A- | CD8α | CD8α | ICOS | CD3ζ |
| | | V158 | | | | |
| 49 | CD8α | CD16A- | CD8α | CD8α | CD27 | CD3ζ |
| | | V158 | | | | |
| 50 | CD8α | CD16A- | CD8α | CD8α | GITR | CD3ζ |
| | | V158 | | | | |
| 51 | CD8α | CD16A- | CD8α | CD8α | HVEM | CD3ζ |
| | | V158 | | | | |
| 52 | CD8α | CD16A- | CD8α | CD8α | TIM1 | CD3ζ |
| | | V158 | | | | |
| 53 | CD8α | CD16A- | CD8α | CD8α | LFA1 | CD3ζ |
| | | V158 | | | (CD11a) | |
| 54 | CD8α | CD16A- | CD8α | CD8α | CD2 | CD3ζ |
| | | V158 | | | | |
| 55 | CD8α | CD16A- | CD8α | FcεR1γ | 4-1BB | FcεR1γ |
| | | V158 | | | (CD137) | |
| 56 | CD8α | CD16A- | CD8α | CD8α | 4-1BB | FcεR1γ |
| | | V158 | | | (CD137) | |

Amino acid sequences of the example chimeric receptors are provided below (signal peptide italicized).

SEQ ID NO: 1:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 2:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIISFFLALTSTALLFLLFFLTLRFSVVKRGKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 3:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDFWVLVVVGGVLACYSLLVTVAFIIFWVRSKKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 4:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLIALVTSGALLAVLGITGYFLMNRKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 5:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLLAALLALLAALLALLAALLARSKKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 6:
MALPVTALLLPLALLLHAARPQAAAPPKAVLKLEPPWINVLQEDSVTLTC
QGARSPESDSIQWFHNGNLIPTHTQPSYRFKANNNDSGEYTCQTGQTSLS
DPVHLTVLSEWLVLQTPHLEFQEGETIMLRCHSWKDKPLVKVTFFQNGKS
QKFSHLDPTFSIPQANHSHSGDYHCTGNIGYTLFSSKPVTITVQVPSMGS
SSPMGTTTPAPRPPTPAPTIASQPLSLRPEACRPAAGGAVHTRGLDFACD
IYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR
SEQ ID NO: 7:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRLLHSDYMNMTPR
RPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 8:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCALYLLRRDQRLPPDAHKP
PGGGSFRTPIQEEQADAHSTLAKIRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 9:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRLLHSDYMNMTPR
RPGPTRKHYQPYAPPRDFAAYRSKRGRKKLLYIFKQPFMRPVQTTQEEDG
CSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPR
SEQ ID NO: 10:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCKRGRKKLLYIFKQ
PFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR
SEQ ID NO: 11:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGSPAGSPTSTEEGTSESATPESGPGTSTEPSEGSAP
GSPAGSPTIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 12:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRGGHSDYMNMTPR
RPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 13:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRGGHSDYMNMTPR
RPGPTRKHYQPYAPPRDFAAYRSKRGRKKLLYIFKQPFMRPVQTTQEEDG
CSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPR
SEQ ID NO: 14:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDMALIVLGGVAGLLLFIGLGIFFCVRKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 15:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDMALIVLGGVAGLLLFIGLGIFFCVRRSKRSRGGHSDYMNMTP
RRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 16:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLCYILDAILFLYGIVLTLLYCRLKKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 17:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLLLILLGVLAGVLATLAALLARSKKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 18:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDITLGLLVAGVLVLLVSLGVAIHLCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 19:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDVSFCLVMVLLFAVDTGLYFSVKTNKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 20:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDVAAILGLGLVLGLLGPLAILLALYKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 21:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLCYLLDGILFIYGVILTALFLRVKKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 22:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDVMSVATIVIVDICITGGLLLLVYYWSKNRKRGRKKLLYIFKQ
PFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR
SEQ ID NO: 23:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDGFLFAEIVSIFVLAVGVYFIAGQDKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 24:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDGIIVTDVIATLLLALGVFCFAGHETKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 25:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDVIGFRILLLKVAGFNLLMTLRLWKRGRKKLLYIFKQPFMRPV
QTTQEEDGCSCRFPEEEEGGCELRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 26:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIIVAVVIATAVAAIVAAVVALIYCRKKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 27:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDVLFYLAVGIMFLVNTVLWVTIRKEKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 28:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIIILVGTAVIAMFFWLLLVIILRTKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 29:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDLGWLCLLLLPIPLIVWVKRKKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR
SEQ ID NO: 30:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIAIYCIGVFLIACMVVTVILCRMKKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 31:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLFGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 32:
MALPVTALLLPLALLLHAARPQVDTTKAVITLQPPWVSVFQEETVTLHCE
VLHLPGSSSTQWFLNGTATQTSTPSYRITSASVNDSGEYRCQRGLSGRSD
PIQLEIHRGWLLLQVSSRVFTEGEPLALRCHAWKDKLVYNVLYYRNGKAF
KFFHWNSNLTILKTNISHNGTYHCSGMGKHRYTSAGISVTVKELFPAPVL
NASVTSPLLEGNLVTLSCETKLLLQRPGLQLYFSFYMGSKTLRGRNTSSE
YQILTARREDSGLYWCEAATEDGNVLKRSPELELQVLGLQLPTPVWFHIY
IWAPLAGTCGVLLLSLVITLYCKRGRKKLLYIFKQPFMRPVQTTQEEDGC
SCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGH
DGLYQGLSTATKDTYDALHMQALPPR
SEQ ID NO: 33:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKIYIWA
PLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPR
SEQ ID NO: 34:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQEPKSCDKTHTCPGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKIYIWAPLAGTCGVLLLSL
VITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
SEQ ID NO: 35:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQEPKSCDKTHTCPIYIWAPLAGTCGVLLLSLVITLYCK
RGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
SEQ ID NO: 36:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEAFACDIYIWAPL
AGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR
SEQ ID NO: 37:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPFACDIYIWAPLAGTCGVLLLSLVITL
YCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHM
QALPPR
SEQ ID NO: 38:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGGGSGGGGSGGGGSIYIWAPLAGTCGVLLLSLVITL
YCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHM
QALPPR
SEQ ID NO: 39:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGGGSGGGGSGGGGSGGGGSGGGGSGGGGSIYIWAPL
AGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR
SEQ ID NO: 40:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGGGSGGGGSGGGGSGGGGSGGGGSGGGGSGGGGSGG
GGSGGGGSIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 41:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGGGSGGGGSGGGGSGGGGSGGGGSGGGGSGGGGSGG
GGSGGGGSGGGGSGGGGSGGGGSIYIWAPLAGTCGVLLLSLVITLYCKRG
RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
SEQ ID NO: 42:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGSPAGSPTSTEEGTSESATPESGPGTSTEPSEGSAP
GSPAGSPTSTEEGTSTEPSEGSAIYIWAPLAGTCGVLLLSLVITLYCKRG
RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
SEQ ID NO: 43:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGSPAGSPTSTEEGTSESATPESGPGTSTEIYIWAPL
AGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR
SEQ ID NO: 44:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGGSPAGSPTSTEEGTIYIWAPLAGTCGVLLLSLVITL
YCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHM
QALPPR
SEQ ID NO: 45:
MLRLLLALNLFPSIQVTGGMRTEDLPKAVVFLEPQWYRVLEKDSVTLKCQ
GAYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKGR
KYFHHNSDFYIPKATLKDSGSYFCRGLVGSKNVSSETVNITITQGLAVST
ISSFFPPGYQTTTPAPRPPTPAPTIASQPLSLRPEACRPAAGGAVHTRGL
DFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 46:
METDTLLLWVLLLWVPGSTGDGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 47:
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPR
SEQ ID NO: 48:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCCWLTKKKYSSSVHDPNGE
YMFMRAVNTAKKSRLTDVTLRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR
SEQ ID NO: 49:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCQRRKYRSNKGESPVEPAE
PCRYSCPREEEGSTIPIQEDYRKPEPACSPRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 50:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCQLGLHIWQLRSQCMWPRE
TQLLLEVPPSTEDARSCQFPEEERGERSAEEKGRLGDLWVRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR
SEQ ID NO: 51:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCCVKRRKPRGDVVKVIVSV
QRKRQEAEGEATVIEALQAPPDVTTVAVEETIPSFTGRSPNHRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR
SEQ ID NO: 52:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKKYFFKKEVQQLSVSFSS
LQIKALQNAVEKEVQAEDNIYIENSLYATDRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 53:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCYKVGFFKRNLKEKMEAGR
GVPNGIPAEDSEQLASGQEAGDPGCLKPLHEKDSESGGGKDRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
SEQ ID NO: 54:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRKKQRSRRNDEELETRA
HRVATEERGRKPHQIPASTPQNPATSQHPPPPPGHRSQAPSHRPPPPGHR
VQHQPQKRPPAPSGTQVHQQKGPPLPRPRVQPKPPHGAAENSLSPSSNRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR
SEQ ID NO: 55:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDPQLCYILDAILFLYGIVLTLLYCRLKIQVRKAAITSYEKSDG
VYTGLSTRNQETYETLKHEKPPQKRGRKKLLYIFKQPFMRPVQTTQEEDG
CSCRFPEEEEGGCEL
SEQ ID NO: 56:
MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRLKIQVRKAAITSYEKSDGVYTGLST
RNQETYETLKHEKPPQ

In some embodiments, the ACTR polypeptides described herein may comprise a CD16A extracellular domain with binding affinity and specificity for the Fc portion of an immunoglobulin, a transmembrane domain, and a CD3ζ cytoplasmic signaling domain. Such an ACTR polypeptide may further include one or more co-stimulatory signaling domains, at least one of which is a CD28 co-stimulatory signaling domain. The ACTR polypeptides are configured such that, when expressed on a host cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling. In some examples, an ACTR polypeptide as described herein may comprise, from N-terminus to C-terminus, the Fc binding domain such as a CD16A extracellular domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

In some embodiments, if the transmembrane domain of the ACTR polypeptide is a CD8 transmembrane domain, the ACTR polypeptide may be free of a hinge domain from any non-CD16A receptor or contain a shortened hinge domain. Alternatively or in addition, the ACTR polypeptide may comprise more than one co-stimulatory signaling domain.

In other embodiments, the ACTR polypeptide described herein may have no non-CD16A hinge domain, or contain no hinge domain at all. In yet other embodiments, the ACTR polypeptide described herein may have a shortened hinge domain (e.g., including up to 25 amino acid residues).

Alternatively or in addition, the ACTR polypeptides described herein may contain two or more co-stimulatory signaling domains, which may link to each other or be separated by the ITAM-containing cytoplasmic signaling domain. The extracellular Fc binder, transmembrane domain, optional co-stimulatory signaling domain(s), and ITAM-containing cytoplasmic signaling domain in an ACTR polypeptide may be linked to each other directly, or via a peptide linker. In some embodiments, any of the ACTR polypeptides described herein may comprise a signal sequence at the N-terminus.

Additional examples of the ACTR polypeptides described herein are provided below:

ACTR polypeptide

SEQ ID NO: 141

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCQRRKYRSNKGESPVEPAE
PCHYSCPREEEGSTIPIQEDYRKPEPACSPRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
ACTR polypeptide

SEQ ID NO: 142

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQFWVLVVVGGVLACYSLLVTVAFIIFWVRSKRSRLLHS
DYMNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR
ACTR polypeptide

SEQ ID NO: 143

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKKKYSSSVHDPNGEYMFM
RAVNTAKKSRLTDVTLRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR
ACTR polypeptide

SEQ ID NO: 144

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRRDQRLPPDAHKPPGGGS
FRTPIQEEQADAHSTLAKIRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPR
ACTR polypeptide

SEQ ID NO: 145

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRLLHSDYMNMTPR
RPGPTRKHYQPYAPPRDFAAYRSQRRKYRSNKGESPVEPAEPCHYSCPRE
EEGSTIPIQEDYRKPEPACSPRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR
ACTR polypeptide

SEQ ID NO: 146

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRLLHSDYMNMTPR
RPGPTRKHYQPYAPPRDFAAYRSKKKYSSSVHDPNGEYMFMRAVNTAKKS
RLTDVTLRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR
ACTR polypeptide

SEQ ID NO: 147

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCRSKRSRLLHSDYMNMTPR
RPGPTRKHYQPYAPPRDFAAYRSRRDQRLPPDAHKPPGGGSFRTPIQEEQ
ADAHSTLAKIRVKFSRSADAPAYQQGQNQLYNELNLGRREEYDVLDKRRG
RDPEMGGKPRRKNPQEGLYNELQKDKMAEAYSEIGMKGERRRGKGHDGLY
QGLSTATKDTYDALHMQALPPR
ACTR polypeptide

SEQ ID NO: 148

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRSKRSRLLHSDYMNMTPRRPGPTRKH
YQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPR
ACTR polypeptide

SEQ ID NO: 149

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR
ACTR polypeptide

SEQ ID NO: 150

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCRSKRSRLLHSDYM
NMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
ACTR polypeptide

SEQ ID NO: 151

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCQRRKYRSNKGESP
VEPAEPCHYSCPREEEGSTIPIQEDYRKPEPACSPRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
ACTR polypeptide

SEQ ID NO: 152

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCRSKRSRLLHSDYM
NMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
ACTR polypeptide

SEQ ID NO: 153

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCKKKYSSSVHDPNG
EYMFMRAVNTAKKSRLTDVTLRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR
ACTR polypeptide

SEQ ID NO: 154

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQIYIWAPLAGTCGVLLLSLVITLYCRRDQRLPPDAHKP
PGGGSFRTPIQEEQADAHSTLAKIRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR polypeptide

SEQ ID NO: 155

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQFACDIYIWAPLAGTCGVLLLSLVITLYCKRGRKKLLY
IFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPR
ACTR polypeptide

SEQ ID NO: 156

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQFACDIYIWAPLAGTCGVLLLSLVITLYCRSKRSRLLH
SDYMNMTPRRPGPTRKHYQPYAPPRDFAAYRSRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR
ACTR polypeptide

SEQ ID NO: 157

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDFWVLVVVGGVLACYSLLVTVAFIIFWVRSKRSRLLHSDYMNM
TPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR
ACTR polypeptide

SEQ ID NO: 158

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR
ACTR polypeptide

SEQ ID NO: 159

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQGKHLCPSPLFPGPSKPFWVLVVVGGVLACYSLLVTVA
FIIFWVRSKRSRLLHSDYMNMTPRRPGPTRKHYQPYAPPRDFAAYRSRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR

In some embodiments, the ACTR polypeptide described herein may contain a mutated Fc receptor extracellular domain as described herein (ACTR variants). Examples of such ACTR variants are provided below:

ACTR variant

SEQ ID NO: 57

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 58

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 59

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 60

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHRVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 61

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHMVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 62

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHLVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 63

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLWGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 64

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLDGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 65

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLKGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 66

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVFSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 67

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVWSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 68

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFYHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 69

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFFHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 70

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFWHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 71

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKWFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 72

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKAFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 102

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 103

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSKAVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 104

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKYFHHNSDFYIPKATLKDSGSYFCRGLVGSDNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 105

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKYFHHNSDFYIPKATLKDSGSYFCRGLVGSDQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 106

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKYFHHNSDFYIPKATLKDSGSYFCRGLVGSENVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 107

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKYFHHNSDFYIPKATLKDSGSYFCRGLVGSEQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 108

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSDNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 109

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSENVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 110

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSDQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 111

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLVGSEQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 112

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKAFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 113

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKYFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 114

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKYFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 115

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKAFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 116

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHNVTYLQNG
KGRKYFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 117

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHNVTYLQNG
KGRKYFHHNSDFYIPKATLKDSGSYFCRGLVGSKQVSSETVNITITQGLA
VSTISSFFPPGYQTTTPAPRPPTPAPTIASQPLSLREACRPAAGGAVHTP
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 118

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKAFHHNSDFYIPKATLKDSGSYFCRGLWGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 119

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKAFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 120

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKAFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 121

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHEVTYLQNG
KGRKAFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 122

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKAFHHNSDFYIPKATLKDSGSYFCRGLWGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 123

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHDVTYLQNG
KGRKAFHHNSDFYIPKATLKDSGSYFCRGLWGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 124

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLNGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 125

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLQGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 126

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKYFHHNSDFYIPKATLKDSGSYFCRGLWGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 127

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHMVTYLQNG
KGRKYFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 128

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHLVTYLQNG
KGRKYFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 129

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHKVTYLQNG
KGRKWFHHNSDFYIPKATLKDSGSYFCRGLVGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 130

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGKLLLQAPRWVFKEEDPIHLRCHSWKNTALHKVTYLQNG
KGRKYFHHNSDFYIPKATLKDSGSYFCRGLDGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 131

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGRLLLQAPRWVFKEEDPIHLRCHSWKNTALHKVTYLQNG
KGRKYFHHNSDFYIPKATLKDSGSYFCRGLDGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 132

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGFLLLQAPRWVFKEEDPIHLRCHSWKNTALHKVTYLQNG
KGRKYFHHNSDFYIPKATLKDSGSYFCRGLDGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 133

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGVLLLQAPRWVFKEEDPIHLRCHSWKNTALHKVTYLQNG
KGRKYFHHNSDFYIPKATLKDSGSYFCRGLDGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 134

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGKLLLQAPRWVFKEEDPIHLRCHSWKNTALHKVTYLQNG
KGRKYFHHNSDFYIPKATLKDSGSYFCRGLDGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 135

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGRLLLQAPRWVFKEEDPIHLRCHSWKNTALHKVTYLQNG
KGRKYFHHNSDFYIPKATLKDSGSYFCRGLDGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 136

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGFLLLQAPRWVFKEEDPIHLRCHSWKNTALHKVTYLQNG
KGRKYFHHNSDFYIPKATLKDSGSYFCRGLDGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 137

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGVLLLQAPRWVFKEEDPIHLRCHSWKNTALHKVTYLQNG
KGRKYFHHNSDFYIPKATLKDSGSYFCRGLDGSKQVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 138

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHMVTYLQNG
KGRKYFHHNSDFYIPKATLKDSGSYFCRGLWGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 139

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHMVTYLQNG
KGRKAFHHNSDFYIPKATLKDSGSYFCRGLVGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
ACTR variant

SEQ ID NO: 140

MALPVTALLLPLALLLHAARPGMRTEDLPKAVVFLEPQWYRVLEKDSVTL
KCQGAYSPEDNSTQWFHNESLISSQASSYFIDAATVDDSGEYRCQTNLST
LSDPVQLEVHIGWLLLQAPRWVFKEEDPIHLRCHSWKNTALHMVTYLQNG
KGRKAFHHNSDFYIPKATLKDSGSYFCRGLWGSKNVSSETVNITITQGLA
VSTISSFFPPGYQTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR

Any of the chimeric receptors described herein can be prepared by a routine method, such as recombinant technology. Methods for preparing the chimeric receptors herein involve generation of a nucleic acid that encodes a polypeptide comprising each of the domains of the chimeric receptors, including the Fc binding, the transmembrane domain, at least one co-stimulatory signaling domain, and the cytoplasmic signaling domain. In some embodiments, the nucleic acid also encodes a hinge domain between the extracellular ligand-binding domain of an Fc receptor and the transmembrane domain. The nucleic acid encoding the chimeric receptor may also encode a signal sequence. In some embodiments, the nucleic acid sequence encodes any one of the exemplary chimeric receptors provided by SEQ ID NO: 1-159.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Mutation of one or more residues, for example one or more residues within the extracellular ligand-binding domain that are involved in interaction of the Fc receptor with an antibody, may be made in the nucleic acid sequence encoding said domain prior to or after joining the sequences of each of the components. Alternatively, the nucleic acid encoding the chimeric receptor may be synthesized. In some embodiments, the nucleic acid is DNA. In other embodiments, the nucleic acid is RNA.

Nucleic acids encoding any of the ACTR constructs may be inserted into a suitable vector for expressing in suitable immune cells, such as T cells or NK cells. Non-limiting examples of useful vectors of the disclosure include viral vectors such as, e.g., retroviral vectors including gamma retroviral vectors, adeno-associated virus vectors (AAV vectors), and lentiviral vectors.

### II. Immune Cells Expressing Chimeric Receptors

Host cells expressing the chimeric receptors described herein provide a specific population of cells that can recognize target cells bound by bipartite targeting ligand (e.g., antibodies specific for a T cell epitope complexed with a MHC molecule or soluble TCR-Fc-fusion proteins). Engagement of the extracellular ligand-binding domain of a chimeric receptor construct expressed on such host cells (e.g., immune cells) with the Fc portion of a bipartite targeting ligand containing an Fc domain transmits an activation signal to the co-stimulatory signaling domain(s) and the cytoplasmic signaling domain of the chimeric receptor construct, which in turn activates cell proliferation and/or effector functions of the host cell, such as ADCC effects triggered by the host cells. The combination of co-stimulatory signaling domain(s) and the cytoplasmic signaling domain may allow for robust activation of multiple signaling pathways within the cell.

In some embodiments, the host cells are immune cells, such as T cells, NK cells, macrophages, neutrophils, eosinophils, or any combination thereof. In some embodiments, the immune cells are T cells. In some embodiments, the immune cells are NK cells. In other embodiments, the immune cells can be established cell lines, for example, NK-92 cells.

The population of immune cells can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs. In some embodiments, the population of immune cells is derived from a human cancer patient, such as from the bone marrow or from a tumor in a human cancer patient. In some embodiments, the population of immune cells is derived from a healthy donor. The type of host cells desired (e.g., immune cells such as T cells, NK cells, macrophages, neutrophils, eosinophils, or any combination thereof) may be expanded within the population of cells obtained by co-incubating the cells with stimulatory molecules, for example, anti-CD3 and anti-CD28 antibodies may be used for expansion of T cells.

To construct the immune cells that express any of the chimeric receptor constructs described herein, expression vectors for stable or transient expression of the chimeric receptor construct may be constructed via conventional methods as described herein and introduced into immune host cells. For example, nucleic acids encoding the chimeric receptors may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. See section VI below. Suitable vectors and methods for producing vectors containing transgenes are well known and available in the art. Examples of the preparation of vectors for expression of chimeric receptors can be found, for example, in US2014/0106449, herein incorporated by reference in its entirety.

In some embodiments, the chimeric receptor construct or the nucleic acid encoding said chimeric receptor is a DNA molecule. In some embodiments, the chimeric receptor construct or the nucleic acid encoding said chimeric receptor is a transposon. In some embodiments, the chimeric receptor construct or the nucleic acid encoding said chimeric receptor is a plasmid. In some embodiments, chimeric receptor construct or the nucleic acid encoding said chimeric receptor is a DNA plasmid may be electroporated to immune cells (see, e.g., Till, et al. Blood (2012) 119(17): 3940-3950). In some embodiments, the nucleic acid encoding the chimeric receptor is an RNA molecule, which may be electroporated to immune cells.

Any of the vectors comprising a nucleic acid sequence that encodes a chimeric receptor construct described herein is also within the scope of the present disclosure. Such a vector may be delivered into host cells such as host immune cells by a suitable method. Methods of delivering vectors to immune cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6): 2082-2087); or viral transduction. In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses. In some embodiments, the vectors for expression of the chimeric receptors are gamma-retroviruses. In some embodiments, the vectors for expression of the chimeric receptors are adeno-associated viruses.

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

Following introduction into the host cells a vector encoding any of the chimeric receptors provided herein, the cells are cultured under conditions that allow for expression of the chimeric receptor. In examples in which the nucleic acid encoding the chimeric receptor is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the chimeric receptor is expressed will be evident to one of skill in the art and may be assessed by any known method, for example, detection of the chimeric receptor-encoding mRNA by quantitative reverse transcriptase PCR (qRT-PCR) or detection of the chimeric receptor protein by methods including Western blotting, fluorescence microscopy, and flow cytometry. Alternatively, expression of the chimeric receptor may take place in vivo after the immune cells are administered to a subject.

Alternatively, expression of a chimeric receptor construct in any of the immune cells disclosed herein can be achieved by introducing RNA molecules encoding the chimeric receptor constructs. Such RNA molecules can be prepared by in vitro transcription or by chemical synthesis. The RNA molecules can then be introduced into suitable host cells such as immune cells (e.g., T cells, NK cells, macrophages, neutrophils, eosinophils, or any combination thereof) by, e.g., electroporation, transfection reagents, viral transduction or mechanical deformation of cells. For example, RNA molecules can be synthesized and introduced into host immune cells following the methods described in Rabinovich et al., Human Gene Therapy, 17:1027-1035 and WO WO2013/040557.

Methods for preparing host cells expressing any of the chimeric receptors described herein may also comprise activating the host cells ex vivo. Activating a host cell means stimulating a host cell into an activate state in which the cell may be able to perform effector functions (e.g., ADCC). Methods of activating a host cell will depend on the type of host cell used for expression of the chimeric receptors. For example, T cells may be activated ex vivo in the presence of one or more molecule such as an anti-CD3 antibody, an anti-CD28 antibody, IL-2, IL-17, IL-15, or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and K562 cells. In some embodiments, the host cells expressing any of the chimeric receptors described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

The methods of preparing host cells expressing any of the chimeric receptors described herein may comprise expanding the host cells ex vivo. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptors described herein are expanded ex vivo prior to administration to a subject.

In some embodiments, the host cells expressing the chimeric receptors are expanded and activated ex vivo prior to administration of the cells to the subject.

### III. Bipartite Targeting Ligands

Immune cells expressing any of the chimeric receptors described herein can be co-used with a bipartite targeting ligand that binds to the chimeric receptor to achieve the intended therapeutic effects as also described herein. As used herein, a “bipartite targeting ligand” refers a protein that recognizes and binds to a specific T cell epitope complexed with an MHC molecule on the surface of a cell (e.g., of an antigen-presenting cell), and capable of binding to any of the chimeric receptors described herein, which can be expressed on the surface of a suitable immune cell. A bipartite targeting ligand comprises at least two portions: a first portion (e.g., an antibody variable region or a TCR variable region) that binds a T cell epitope of interest complexed with an MHC, and a second portion which is an immunoglobulin Fc domain. Examples include, but are not limited to, antibodies specific to peptide (T cell epitope)/MHC complex (also known as TCR-like antibodies) and a soluble derivative of a T cell receptor (TCR), such as a TCR-Fc fusion protein.

In general, the majority of human TCRs are heterodimer proteins comprising an alpha chain and a beta chain, each of which comprises a variable region and a constant region. A minority of human TCRs are heterodimer proteins comprising a gamma chain and a delta chain, each of which comprises a variable region and a constant region. The variable regions of a TCR are capable of binding specifically to a complex of its cognate T cell epitope presented by an MHC molecule (e.g., HLA molecules in humans). Similar to antibodies, the specificity of a T cell receptor to a T cell epitope-MHC complex is determined by the three complementarity determining regions (CDRs) contained within each of the variable domains. Without wishing to be bound by any particular theory, it is thought that the CDR1 and CDR3 of each of the variable region of the alpha chain and the variable region of the beta chain are responsible for binding residues of the T cell epitope, whereas CDR2 of each of the variable region of the alpha chain and the variable region of the beta chain are responsible for binding the MHC molecule.

As used herein, a “T cell epitope” refers to an antigen fragment (e.g., a peptide epitope or a non-peptidic epitope such as a lipid epitope) that can be bound to a MHC molecule and presented on the surface of a cell (e.g., an antigen presenting cell). T cell epitopes may be either peptides of a target antigen or non-peptide epitopes associated with a target antigen. In general, T cell epitopes that are bound to an MHC Class I molecule are typically about 8-11 amino acids in length. Such T cell epitopes are referred to as MHC Class I-restricted epitopes. T cell epitopes that are bound to an MHC Class II molecule are typically 13-17 amino acids in length, or longer and are referred to as MHC Class II-restricted epitopes. In some embodiments, the MHC-bound T cell epitopes are MHC Class I-restricted epitopes. In other embodiments, the MHC-bound T cell epitopes are MHC Class II-restricted epitopes.

In some embodiments, the T cell epitope is presented (bound) to a human MHC molecule referred to as a Human Leukocyte Antigen (HLA) molecule. In general, HLA molecules are very polymorphic within the human population. In some embodiments, the HLA molecule is HLA-A, HLA-B, HLA-C, HLA-DP, HLA-DM, HLA-DOA, HLA-DOB, HLA-DQ, or HLA-DR. In some embodiments, the HLA molecule is HLA-A*02:01, HLA-A*02:03, HLA-A*02:06, HLA-A*03:01, HLA-A*11:01, HLA-A*23:01, HLA-A*24:02, HLA-A*26:01, HLA-A*30:01, HLA-A*30:02, HLA-A*31:01, HLA-A*32:01, HLA-A*33:01, HLA-A*68:01, HLA-A*68:02, HLA-B*07:02, HLA-B*08:01, HLA-B*15:01, HLA-B*35:01, HLA-B*40:01, HLA-B*44:02, HLA-B*44:03, HLA-B*51:01, HLA-B*53:01, HLA-B*57:01, HLA-B*2705, HLA-B*58:01, HLA-DR4, and HLA-DQ8. In some embodiments, the HLA molecule is HLA-A*02:01, also referred to as HLA-A0201.

Any MHC-bound T cell epitope can be a target of a bipartite targeting ligand described herein. T cell epitopes of a target antigen can be predicted by methods known in the art, for example, using prediction algorithms such as those provided by tools.iedb.org/main/tcell, omicotools.com/t-cell-epitopes-category, www.proimmune.com/ecommerce/page.php?page=prediction, and www.syfpeithi.de/bin/MHCServer.dll/EpitopePrediction.htm.

Examples of target antigens from which a T cell epitope may be derived include, without limitation, CD17/L1-CAM, CD19, CD20, CD22, CD30, CD33, CD37, CD52, CD56, CD70, CD79b, CD138, CEA, DS6, EGFR, EGFRvIII, ENPP3, FR, GD2, GPNMB, HER2, IL-13Rα2, Mesothelin, MUC1, MUC16, Nectin-4, PSMA, SCL44A4, WT-1, HA-1H, NY-ESO-1, Human Papillomavirus (HPV) E6, HPV E7, Epstein-Barr Virus (EBV) LMP2, Human Immunodeficiency Virus gag protein, and heat-shock proteins (HSP), such as HSP70.

Table 6 includes examples of MHC-bound T cell epitopes that may be bound by the bipartite targeting ligands.

TABLE 6
Exemplary T Cell Epitopes

| Target antigen | T cell epitope |
|---|---|
| WT-1 | RMFPNAPYL (SEQ ID NO: 75) |
| NY-ESO-1₁₅₇₋₁₆₅ | SLLMWITQC (SEQ ID NO: 76) |
| HA-1H | VLHDDLLEA (SEQ ID NO: 77) |
| HPV16 E7 | YMLDLQPETT (SEQ ID NO: 91) |
| HPV16 E6 | TIHDIILECV (SEQ ID NO: 92) |
| EBV LMP2 | CLGGLLTMV (SEQ ID NO: 93) |
| EBV LMP2 | FLYALALLL (SEQ ID NO: 94) |
| HIV gag TW10 peptide | TSTLQEQIGW (SEQ ID NO: 95) |
| HIV gag KK10 peptide | KRWIILGLNK (SEQ ID NO: 96) |
| HSP 70 B29 peptide | VLRCINEPTAAALAY |
| | (SEQ ID NO: 97) |
| HSP 70 B29 peptide | VLRIINEPTAAAIAY |
| | (SEQ ID NO: 98) |
| HSP 70 B29 peptide | VMRIVNEPTAAAIAY |
| | (SEQ ID NO: 99) |
| HSP 70 B29 peptide | VLRIVNEPTAAALAY |
| | (SEQ ID NO: 100) |

In some embodiments, the bipartite targeting ligand binds the T cell epitope RMFPNAPYL (SEQ ID NO: 75) complexed with HLA-A*02:01. In some embodiments, the bipartite targeting ligand binds the T cell epitope SLLMWITQC (SEQ ID NO: 76) complexed with HLA-A*02:01. In some embodiments, the bipartite targeting ligand binds the T cell epitope VLHDDLLEA (SEQ ID NO: 77) complexed with HLA-A*02:01. In other examples, the bipartite targeting ligand binds any of the HPV or EBV epitopes listed in Table 6 above complexed with HLA-A*02:01. In some examples, the bipartite targeting ligand binds the T cell epitope TSTLQEQIGW (SEQ ID NO: 95) complexed with HLA-B*57. In some examples, the bipartite targeting ligand binds the T cell epitope KRWIILGLNK (SEQ ID NO: 96) complexed with HLA-B*2705. In some examples, the bipartite targeting ligand binds a T cell epitope of a heat-shock protein (HSP), such as the B29 peptide of HSP 70, provided by any of the HSP70 sequences listed in Table 6 complexed with HLA-DQ8.

In some embodiments, the bipartite targeting ligand preferentially binds the T cell epitope when complexed with a MHC molecule, but does not substantially bind the T cell epitope in the absence of the MHC molecule or the MHC molecule in the absence of the T cell epitope. Antibodies that recognize and bind the MHC-restricted TCR epitope provided by SEQ ID NO: 75 derived from WT-1, the MHC-restricted TCR epitope provided by SEQ ID NO: 76 derived from NY-ESO-1, the MHC-restricted TCR epitope of HA-1H provided by SEQ ID NO: 77, are known in the art, see, e.g., Veomett, et al. Clin. Cancer Res. (2014) 20(15): 4036-4046; Held et al. Eur. J. Immunol. (2004) 34: 2919-2929; and Inaguma et al. Gene Ther (2014) 21: 575-584, respectively. TCRs that recognize and bind the MHC restricted TCR epitope provided by SEQ ID NO: 95 or SEQ ID NO: 96, are known in the art and may be used to generate the bipartite targeting ligands described herein. See, e.g, Chen et al. Nat. Immunol. (2012) 13(7): 691-700.

Any of the bipartite targeting ligands described herein may have a suitable binding affinity for a peptide/MHC complex, and/or a chimeric receptor described herein. For example, the bipartite targeting ligand may have a binding affinity K_{D} of at least 10⁻⁵, 10⁻⁶, 10⁻⁷, 10⁻⁸, 10, 10⁻¹⁰M or lower for a peptide/MHC complex and/or a chimeric receptor. The binding affinity or binding specificity for a bipartite targeting ligand can be determined by a variety of methods including equilibrium dialysis, equilibrium binding, gel filtration, ELISA, surface plasmon resonance, or spectroscopy.

In some embodiments, the bipartite targeting ligand is an antibody that specifically binds a T cell epitope that is complexed with a MHC molecule, e.g., those T cell epitopes noted above, that are complexed with HLA-A*02:01.

An antibody (interchangeably used in plural form) is an immunoglobulin molecule capable of specific binding to a target, such as a peptide/MHC complex, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

Antibodies capable of binding a peptide/MHC complex as described herein can be made by any method known in the art. See, for example, Harlow and Lane, (1988) Antibodies: A Laboratory Manual, Cold Spring Harbor Laboratory, New York. In some embodiments, antibodies specific to a peptide/MHC complex can be made by the conventional hybridoma technology. The peptide/MHC complex, which may be coupled to a carrier protein such as KLH, can be used to immunize a host animal for generating antibodies binding to that complex. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoas say, enzyme immunoassay, or fluorescence immunoassay).

Hybridomas that may be used as source of antibodies encompass all derivatives, progeny cells of the parent hybridomas that produce monoclonal antibodies capable of binding to a peptide/MHC complex. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the peptide/MHC complex. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a peptide/MHC complex can be identified from the library following routine procedures. Positive clones can be subjected to further screening to identify those that bind peptide/MHC complexes.

In other embodiments, the bipartite targeting ligand is a soluble TCR-Fc fusion protein comprising the variable region of a TCR and the Fc region of an antibody, such as an IgG antibody. For example, the TCR-Fc fusion protein may comprise two chains, one comprising the variable region of one chain (e.g., an alpha chain) of a TCR and one or more domains of the Fc region and the other comprising the variable region of the other chain (e.g., beta chain) of a TCR and either variable region or both may be fused to an Fc region. In other embodiments, the TCR-like Fc fusion protein may be a single chain protein comprising one or more variable regions that recognize and bind to an MHC-restricted T cell epitope and an Fc fragment. See, e.g., Belmont et al. Cancer Res. 64: 503. In yet other examples, the soluble TCR-Fc fusion protein is a single-chain polypeptide comprising a variable domain of a TCR chain (e.g., an alpha chain or a beta chain) fused to an Fc fragment.

The Fc-containing TCR-fusion protein may be prepared by any methods known in the art, e.g., recombinant technology.

Alternatively or in addition, antibodies and TCR-fusion proteins described herein may be generated through a display library selection. Briefly, an oligonucleotides encoding a library of TCRs, antibodies, or portions thereof, can be fused to a cellular or phage protein such as bacteriophage pIII coat protein and expressed on the surface of cells or phage particles. The display libraries expressing the TCR-fusion proteins, antibodies, or portions thereof (e.g. TCR variable region, or Fab fragment) may undergo one or more rounds of positive selection in which cells or phage particles that bind to the desired T cell epitope complexed to an MHC molecule are isolated. The isolated cell/phage may also be subjected to one or more rounds of negative selection in which the cell/phage that binds to the T cell epitope in the absence of the MHC molecule, the MHC molecule in the absence of the T cell epitope, and/or a T cell epitope comprising one or more mutations relative to the desired T cell epitope may be discarded. The nucleic acid sequences of the TCR-fusion protein, the antibody, or a portion thereof that is expressed by the isolated cell/phage may also undergo further mutagenesis to achieve an optimized TCR-fusion protein or antibody, for example, for optimal manufacturing, drug properties, and/or desired binding affinity. See, e.g., Skora et al. Proc. Nat. Acad. Sci UDA (2015) 112(32): 9967-9972; Dao et al. Sci Transl Med. (2013) 5(176).

Any bipartite targeting ligand described herein, including an antibody or soluble TCR-Fc fusion protein, may contain a modified Fc domain that is capable of binding to a chimeric receptor containing a mutated Fc receptor extracellular domain or a scFv that specifically binds the modified Fc domain as relative to the wild-type counterpart. Immune cells expressing such chimeric receptors can be co-used with a bipartite targeting ligand comprising a modified Fc domain to enhance the efficacy of the immunotherapy and/or to reduce autoimmune effects. In some embodiments, the bipartite targeting ligand has been modified or mutated relative to its wild-type counterpart, for example, having altered post-translational modification as relative to its wild-type counterpart having the same amino acid sequences.

The design of TCR-Fc fusion proteins that may be used in the methods and kits described herein are known in the art, see e.g. Wu et al. mAbs (2015) 7(2): 364-376 and McCormack et al. Cancer Immunol. Immunother. (2013) 62:773-785.

In some embodiments, the Fc domain of the bipartite targeting ligand comprises one or more mutations relative to the wild-type Fc domain, such a mutation may be referred to as a compensatory mutation, which can be one or more mutations in the Fc domain of the bipartite targeting ligand that restores or allows interaction between the mutated bipartite targeting ligand and the corresponding chimeric receptor variant (ACTR variant) as described herein. The bipartite targeting ligand may comprise one or more mutations in residues of the Fc region involved in the interaction between the Fc region and an Fc binding domain to allow for interaction between the Fc region of the bipartite targeting ligand and the mutated Fc binding domain of an Fc receptor. In some embodiments, the one or more mutations allow for interaction between the bipartite targeting ligand and the mutated Fc binder of a chimeric receptor variant that did not occur in absence of the one or more mutations in the antibody. In some embodiments, the one or more mutations in the Fc domain of the bipartite targeting ligand are located in the hinge and/or CH2 domain of the Fc domain. Examples of mutations in the Fc domain, for example in the Fc domain of antibodies, are known in the art and can be used in the bipartite targeting ligands described herein. Such examples can be found, for example, in U.S. Pat. Nos. 7,601,335, 8,188,231, and 9,120,856, and include substitution mutations of amino acid residues S239, F243, R292, S298, Y300, V305, A330, 1332, E333, K334, or P396 (using EU index numbering as described in Kabat et al., (1991), Sequences of Proteins of Immunological Interest, 5th Ed.). In some embodiments, the one or more mutations in the Fc fragment can be S239D, F243L, R292P, S298A, Y300L, V305I, A330L, I332E, I332D, E333A, K334A, and/or P396L. See, for example, Shields et al. J. Biol. Chem. (2001) 276(9):6591-6604; Lazar et al. Proc. Natl. Acad. Sci. USA (2006) 103(11): 4005-4010; Stavenhagen et al. Cancer Res. (2007) 67(18): 8882-8890; Isoda et al. PLoS One (2015) 10(10): e0140120; Lu et al. J. Immunol. Met. (2011) 365:132-141; Liu et al. J. Biol. Chem. (2014) 289(6): 3571-3590; and Smith et al. Proc. Natl. Acad. Sci. USA (2012) 109(16):6181-6186. See also U.S. Pat. Nos. 6,737,056, 7,662,925, 7,317,091, and 8,217,147. The relevant disclosures of the referenced publications are incorporated by reference for the purposes or subject matter referenced herein.

In some examples, the bipartite targeting ligand comprises a mutated Fc domain as provided below:

(SEQ ID NO: 78)

TCPPCPAPELLGGPDVFLFPPKPKDTLMISRTPEVTCVVVDVSHEDPEVK
FNWYVDGVEVHNAKTKPREEQYNSTYRVVSVLTVLHQDWLNGKEYKCKVS
NKALPLPEEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK

(SEQ ID NO: 79)

TCPPCPAPELLGGPDVFLFPPKPKDTLMISRTPEVTCVVVDVSHEDPEVK
FNWYVDGVEVHNAKTKPREEQYNSTYRVVSVLTVLHQDWLNGKEYKCKVS
NKALPLPEEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK

(SEQ ID NO: 80)

TCPPCPAPELLGGPDVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPEEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK

(SEQ ID NO: 81)

TCPPCPAPELLGGPSVFLLPPKPKDTLMISRTPEVTCVVVDVSHEDPEVK
FNWYVDGVEVHNAKTKPPEEQYNSTLRVVSILTVLHQDWLNGKEYKCKVS
NKALPAPIEKTISKAKGQPREPQVYTLPPSRDELTKNQVSLTCLVKGFYP
SDIAVEWESNGQPENNYKTTPLVLDSDGSFFLYSKLTVDKSRWQQGNVFS
CSVMHEALHNHYTQKSLSLSPGK

An ACTR variant for use in the disclosed compositions and methods may comprise an amino acid substitution at one or more positions corresponding to 122, 134, 160, and 164 in SEQ ID NO: 74 (e.g., CD16A mutant V160Q, CD16A mutant V160W, or CD16A mutant K122L), and the Fc-containing bipartite targeting ligand to be co-used with the ACTR variant may comprise an amino acid substitution at one or more positions corresponding to S239, F243, R292, S298, Y300, V305, A330, 1332, E333, K334, and P396 of a wild-type antibody.

Alternatively or in addition, the bipartite targeting ligands are modified to reduce, eliminate, or add one or more sugar moieties. In some embodiments, the bipartite targeting ligands are afucosylated proteins. The terms “afucosylated” and “non-fucosylated” may be used interchangeably throughout and refer to an protein that has reduced or absent fucosylation. In some embodiments, the bipartite targeting ligands are modified to add one or more additional glycosylation sites. In some embodiments, the bipartite targeting ligands are produced under conditions that result in altered glycosylation of the protein.

It is appreciated in the art, that glycosylation of an Fc domain, particularly residue Asn of the CH2 domains, plays a critical role in the interaction between the Fc domain and an Fc receptor. See, for example, Nose M, et al Proc Natl Acad Sci USA (1983)80:6632-6636. In some embodiments, the Fc domain of the bipartite targeting ligand is not glycosylated at residue Asn297. In some embodiments, the bipartite targeting ligand is an afucosylated protein, for example a protein from which the fucose moieties are not present. In some embodiments, the bipartite targeting ligand comprises mutation of one or more residue in the Fc domain that is glycosylated, thereby resulting in a bipartite targeting ligand that has reduced glycosylation or is not glycosylated.

In some embodiments, the bipartite targeting ligand may be modified after production (e.g., post-translationally or after isolation) to reduce or eliminate the fucose moieties present on the protein.

### IV. Application of Immune Cells Expressing Chimeric Receptor in Immunotherapy

Host cells (e.g., immune cells) expressing the chimeric receptors (the encoding nucleic acids or vectors comprising such) described herein are useful for enhancing ADCC in a subject and/or for enhancing the efficacy of an immunotherapy. As used herein, the term “subject” refers to any mammal, such as a human, monkey, mouse, rabbit, or domestic mammal. In some embodiments, the subject is a human. In some embodiments, the subject is a human cancer patient. In some embodiments, the subject has been treated or is being treated with any of the bipartite targeting ligands, including antibodies and TCR-Fc fusion proteins, described herein.

The immune cells can be mixed with a pharmaceutically acceptable carrier to form a pharmaceutical composition, which is also within the scope of the present disclosure. In some embodiments, the pharmaceutical composition also includes a bipartite targeting ligand, such as an antibody and/or a TCR-Fc fusion protein.

To perform the methods described herein, a therapeutically effective amount of the immune cells expressing any of the chimeric receptor variant constructs described herein and a therapeutically effective amount of a bipartite targeting ligand that binds the chimeric receptor variant can be co-administered to a subject in need of the treatment. As used herein the term “therapeutically effective amount” refers to that quantity of a compound, cell population (e.g., immune cells expressing the chimeric receptors described herein), bipartite targeting ligand, or pharmaceutical composition (e.g., a composition comprising immune cells such as T lymphocytes and/or NK cells) comprising a chimeric receptor of the disclosure, and optionally further comprising a bipartite targeting ligand that binds the chimeric receptor that is sufficient to result in a desired activity upon administration to a subject in need thereof. Within the context of the present disclosure, the term “therapeutically effective amount” refers to that quantity of a compound, cell population, or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination may or may not include amounts of each ingredient that would have been effective if administered individually.

The immune cells expressing the chimeric receptors described herein may be autologous to the subject, i.e., the immune cells are obtained from the subject in need of the treatment, genetically engineered for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered for expression of the chimeric receptor construct, and administered to a second subject that is different from the first subject but of the same species. For example, allogeneic immune cells may be derived from a human donor and administered to a human recipient who is different from the donor.

The T lymphocyte may be an allogeneic T lymphocyte. Such T lymphocytes may be from donors with partially matched HLA subtypes or with epigenetic profiles with reduced chance for inducing graft-versus-host disease. Alternatively, virally-selected T lymphocytes may be used. In some examples, the allogeneic T cells can be engineered to reduce the graft versus host effects. For example, the expression of the endogenous T cell receptor can be inhibited or eliminated. Alternatively or in addition, expression of one or more components of the Major Histocompatibility Complex (MHC) Class I and/or Class II complex (e.g., β-2-microglobulin) can be reduced or eliminated. In other examples, a natural killer cell inhibitory receptor can be expressed on the T lymphocyte.

In some embodiments, the immune cells are administered to a subject in an amount effective in enhancing ADCC activity by least 20%, e.g., 50%, 80%, 100%, 2-fold, 5-fold, 10-fold, 20-fold, 50-fold, 100-fold or more. In some embodiments, the immune cells and the bipartite targeting ligand are administered to a subject in an amount effective in enhancing ADCC activity by least 20%, e.g., 50%, 80%, 100%, 2-fold, 5-fold, 10-fold, 20-fold, 50-fold, 100-fold or more, as compared to the ADCC when the bipartite targeting ligand is administered in the absence of the immune cells expressing the chimeric receptors as described herein.

In some embodiments, the immune cells are co-used with a bipartite targeting ligand (e.g., an antibody or a TCR-Fc fusion protein) so as to enhance the efficacy of the immunotherapy. In general, immunotherapy using bipartite targeting ligands is used to treat, alleviate, or reduce the symptoms of any disease or disorder for which the immunotherapy is considered useful in a subject. In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As described herein, the chimeric receptors comprise an extracellular domain of an Fc receptor that binds to an Fc fragment of a bipartite targeting ligand, as described herein. In some embodiments, the host cells expressing such chimeric receptors are administered in the presence of or in combination with a bipartite targeting ligand. In such therapy, a bipartite targeting ligand may bind to a peptide of a target protein that is presented by a MHC molecule on the cell surface. In some embodiments, the target protein and presence of a peptide of the target protein presented by an MHC molecule on a cell surface indicates that the cell is a target cell for effector function of the immune cells expressing chimeric receptors, as described herein. In some embodiments, the presence of a peptide of the target protein presented by an MHC molecule is associated with a disease indication. In some embodiments, the presence of a peptide of the target protein presented by an MHC molecule on the surface of a cell indicates the cell is in a diseased state (e.g., a target cell), for example that the cell is a cancer cell or the cell is infected with an infectious organism. In some embodiments, the peptide of the target protein differentially presented in MHC molecules on the surface of cells in a diseased state (e.g., target cells) as compared to normal healthy cells (e.g., not presented on non-cancer cells or expressed at a lower level on non-cancer cells).

The efficacy of an immunotherapy using a bipartite targeting ligand may be assessed by any method known in the art and would be evident to a skilled medical professional. For example, the efficacy of the immunotherapy using a bipartite targeting ligand may be assessed by survival of the subject or tumor or cancer burden in the subject or tissue or sample thereof. In some embodiments, the immune cells are administered to a subject in need of the treatment in an amount effective in enhancing the efficacy of an immunotherapy using a bipartite targeting ligand by at least 20%, e.g., 50%, 80%, 100%, 2-fold, 5-fold, 10-fold, 20-fold, 50-fold, 100-fold or more, as compared to the efficacy in the absence of the immune cells.

In some embodiments, the immune cells expressing any of the chimeric receptors disclosed herein are administered to a subject who has been treated or is being treated with an using a bipartite targeting ligand (e.g., an antibody or a TCR-Fc fusion protein). The immune cells expressing any one of the chimeric receptors disclosed herein may be co-administered with a bipartite targeting ligand. For example, the immune cells may be administered to a human subject simultaneously with a bipartite targeting ligand. Alternatively, the immune cells may be administered to a human subject during the course of an immunotherapy using a bipartite targeting ligand. In some examples, the immune cells and a bipartite targeting ligand can be administered to a human subject at least 4 hours apart, e.g., at least 12 hours apart, at least 1 day apart, at least 3 days apart, at least one week apart, at least two weeks apart, or at least one month apart.

To practice the method disclosed herein, an effective amount of the immune cells expressing chimeric receptors, bipartite targeting ligand (e.g., an antibody or a TCR-Fc fusion protein), or compositions thereof can be administered to a subject (e.g., a human patient) in need of the treatment via a suitable route, such as intravenous administration. Any of the immune cells expressing chimeric receptors, bipartite targeting ligands, or compositions thereof may be administered to a subject in an effective amount. As used herein, an effective amount refers to the amount of the respective agent (e.g., the host cells expressing chimeric receptors, bipartite targeting ligands, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human cancer patient.

In some embodiments, the subject is a human patient suffering from a cancer, which can be carcinoma, lymphoma, sarcoma, blastoma, or leukemia. Examples of cancers for which administration of the cells and compositions disclosed herein may be suitable include, for example, lymphoma, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, and thyroid cancer.

In some embodiments, the subject is a human patient suffering from an inflammatory disorder, including acute inflammation or chronic inflammation. In some embodiments, the human patient is suffering from rheumatoid arthritis, multiple sclerosis, or systemic lupus erythematosus.

In some embodiments, the subject is a human patient suffering from an infectious disease, such as a viral infection. Examples of viruses for which administration of the cells and compositions disclosed herein may be suitable include, for example Human Papillomavirus (HPV), Epstein-Barr Virus (EBV), Human Immunodeficiency Virus (HIV), human hepatitis virus such as hepatitis A virus, hepatitis B virus, hepatitis C virus, and hepatitis D virus.

Any of the immune cells expressing chimeric receptors described herein and/or bipartite targeting ligands that bind to the chimeric receptors may be prepared or administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### V. Combination Treatments

The compositions and methods described in the present disclosure may be utilized in conjunction with other types of therapy for cancer, such as chemotherapy, surgery, radiation, gene therapy, and so forth. Such therapies can be administered simultaneously or sequentially (in any order) with the immunotherapy described herein.

When co-administered with an additional therapeutic agent, suitable therapeutically effective dosages for each agent may be lowered due to the additive action or synergy.

The immunotherapies described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapies described herein include without limitation: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadeno sine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some embodiments, radiation or radiation and chemotherapy are used in combination with the antibody-based immunotherapies described herein.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### VI. Kits for Therapeutic Use

The present disclosure also provides kits for use of the chimeric receptors in enhancing immunotherapy using a bipartite targeting ligand. Such kits may include one or more containers comprising a first pharmaceutical composition that comprises any nucleic acid or host cells (e.g., immune cells such as those described herein), and a pharmaceutically acceptable carrier, and a second pharmaceutical composition that comprises a bipartite targeting ligand (e.g., an antibody or a TCR-Fc fusion protein as described herein) and a pharmaceutically acceptable carrier.

In some embodiments, the kit can comprise instructions for use in any of the methods described herein. The included instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the chimeric receptors and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variants as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1: T Lymphocytes Expressing a CD16 Signaling Receptor can be Used to Cause Antibody Dependent Cancer Cell Death

### Materials and Methods

### Cells

Human cell lines are obtained for use in the following cellular assays described below. Peripheral blood samples are obtained from de-identified by-products of platelet donations from healthy adult donors. Mononuclear cells are enriched by centrifugation on Accu-Prep Human Lymphocytes Cell Separation Media (Accurate Chemical & Scientific Corp., Westbury, N.Y.), and cultured with anti-CD3/CD28 beads (Invitrogen, Carlsbad, Calif.) in RPMI-1640 with 10% fetal bovine serum (FBS), antibiotics, 100 IU interleukin (IL)-2 (Roche, Mannheim, Germany) for 3 days. On day 4, T cells are purified by negative selection with a mixture of CD14, CD16, CD19, CD36, CD56, CD123 and CD235α antibodies and magnetic beads (Pan T Cell Isolation Kit II; Miltenyi Biotec, Bergisch Gladbach, Germany) (purity, >98%). Purified T cells are maintained in the above medium, with the addition of 100 IU IL-2 every other day.

Surface expression of CD16 is analyzed by flow cytometry using R-Phycoerythrin conjugated anti-human CD16 (clone B73.1, BD Biosciences Pharmingen, San Diego, Calif.). For Western blotting, 2×10⁷ T cells are lysed in Cellytic M lysis Buffer (Sigma, St Louis, Mo.) containing 1% protease inhibitor cocktail (Sigma) and 1% phosphatase inhibitor cocktail 2 (Sigma). After centrifugation, lysate supernatants are boiled with an equal volume of LDS buffer (Invitrogen, Carlsbad, Calif.) with or without reducing buffer (Invitrogen) and then separated by NuPAGE Novex 12% Bis-Tris Gel (Invitrogen). The proteins are transferred to a polyvinylidene fluoride (PVDF) membrane, which is incubated with a mouse anti-human CD3ζ (clone 8D3; BD eBioscience Pharmingen) and then with a goat anti-mouse IgG horseradish peroxidase-conjugated secondary antibody (Cell Signaling Technology, Danvers, Mass.). Antibody binding is revealed by using the Amersham ECL Prime detection reagent (GE Healthcare; Little Chalfont, United Kingdom).

mRNA Electroporation

The pVAX1 vector (Invitrogen, Carlsbad, Calif.) is used as a template for in vitro mRNA transcription. The CD16V-BB-ζ cDNA is subcloned into EcoRI and XbaI sites of pVAX1. The corresponding mRNA is transcribed in vitro with T7 mScript mRNA production system (CellScript, Madison, Wis.). Shimasaki et al., Cytotherapy. 2012; 14(7):830-40.

For electroporation, the Amaxa Nucleofector (Lonza, Walkersville, Md.) is used; 1×10⁷ of purified T cells activated with 200 IU/mL IL-2 overnight are mixed with 200 μg/mL mRNA in Cell Line Nucleofector Kit V (Lonza; Basel, Switzerland), transferred into the processing chamber, and transfected using the program X-001. Immediately after electroporation, cells are transferred from the processing chamber into a 24-well plate and then cultured in RPMI-1640 with FBS, antibiotics and 100 IU/mL IL-2 (Roche, Mannheim, Germany). See also Shimasaki et al., Cytotherapy, 2012, 1-11.

### Antibody Binding, Cell Conjugation and Cell Proliferation Assays

To measure the chimeric receptors' antibody-binding capacity, T lymphocytes (5×10⁵) transduced with chimeric receptors or a vector containing GFP only are incubated with TCR-like mAb for 30 minutes at 4° C. After washing twice with phosphate buffered saline (PBS), cells are incubated with goat anti-human IgG-PE (Southern Biotechnology Associates, Birmingham, Ala.) for 10 minutes at room temperature, and cell staining is measured using an Accuri C6 flow cytometer (BD Biosciences).

To measure cell proliferation, 1×10⁶ of T cells transduced with the chimeric receptor or mock-transduced are placed in the wells of a 24-well plate (Costar, Corning, N.Y.) in RPMI-1640 with FBS, antibiotics and 50 IU/mL IL-2. Cells are treated with Streck cell preservative (Streck Laboratories, Omaha, Nebr.) to stop proliferation and labeled with TCR-like mAb for 30 min at 4° C. They are added to the wells, at 1:1 ratio with T cells, on days 0, 7, 14 and 21. The number of viable T cells after culture is measured by flow cytometry.

### Cytotoxicity Assays

To test cytotoxicity, target cells are suspended in RPMI-1640 with 10% FBS, labeled with calcein AM (Invitrogen) and plated into 96-well round bottom plates (Costar). T cells are added at various E: T ratio as indicated in Results, and co-cultured with target cells for 4 hours, with or without the TCR like antibodies. At the end of the cultures, cells are collected, resuspended in an identical volume of PBS, and propidium iodide is added. The number of viable target cells (calcein AM-positive, propidium-iodide negative) is counted using a flow cytometer. For adherent cell lines, cytotoxicity is tested using luciferase-labeled target cells. To measure cytotoxicity against the adherent cell lines their luciferase-labeled derivatives are used. After plating for at least 4 hours, T cells are added as described above. After 4 hours of co-culture, the Promega Bright-Glo luciferase reagent (Promega, Madison, Wis.) is added to each well; 5 minutes later, luminescence is measured using a plate reader Biotek FLx800 (BioTek, Tucson, Ariz.) and analyzed with the Gen5 2.0 Data Analysis Software.

### Xenograft Experiments

Cells expressing luciferase are injected intraperitoneally (i.p.; 0.3×10⁶ cells per mouse) in NOD.Cg-Prkdc^{scid} IL2re^{tm1Wjl}/SzJ (NOD/scid IL2RGnull) mice (Jackson Laboratory, Bar Harbor). Groups of mice receive the TCR-like mAb intraperitoneally (i.p.) 4 days after tumor cell inoculation, with or without i.p. injection of human primary T cells on days 5 and 6. T cells are activated with anti-CD3/CD28 beads for 3 days, transduced with the CD16V-BB-ζ receptor, resuspended in RPMI-1640 plus 10% FBS and then injected at 1×10⁷ cells per mouse. A TCR-like antibody injection is repeated weekly for 4 weeks, with no further T lymphocyte injection. All mice receive i.p. injections of 1000-2000 IU of IL-2 twice a week for 4 weeks. A group of mice receive tissue culture medium instead of the TCR-like mAb or T cells.

Tumor engraftment and growth is measured using a Xenogen IVIS-200 system (Caliper Life Sciences, Hopkinton, Mass.). Imaging begins 5 minutes after i.p. injection of an aqueous solution of D-luciferin potassium salt (3 mg/mouse) and photons emitted from luciferase-expressing cells are quantified using the Living Image 3.0 software.

### Example 2: Evaluation of Combined Activity of ACTR-T Cells and Anti-NY-ESO-1/HLA-A2 Antibody

(i) Afucosylated Monoclonal Antibody T1 Recognized both Endogenous and Exogenous Targets

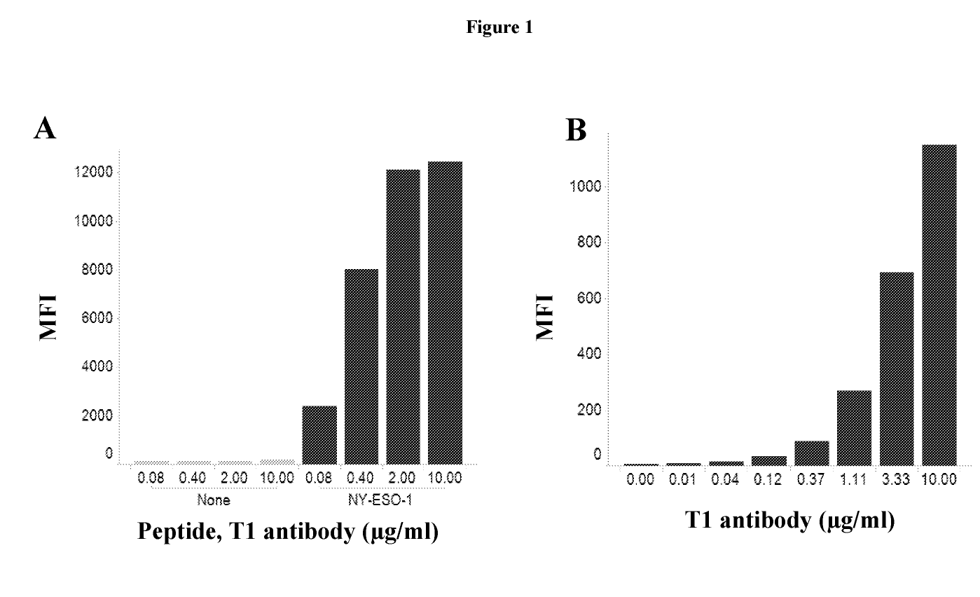

Target cells were T2 cells labeled with and without the NY-ESO-1 analog peptide (SLLMWITQV; SEQ ID NO: 101) (FIG. 1, part A) or U266B1 tumor cells expressing endogenous target (FIG. 1, part B). Samples were stained with a titration of afucosylated monoclonal antibody T1, which is specific to NY-ESO-1/HLA-A2, followed by detection with a fluorescently labeled anti-human secondary antibody. T1 binding was evaluated by flow cytometry and the resulting mean fluorescence intensity (MFI) values are shown in FIG. 1. This experiment demonstrated that afucosylated T1 mAb recognized both exogenous and endogenous NY-ESO-1/HLA-A2 antigen.

(ii) Jurkat NFAT Luciferase Reporter Assay

The ability of different ACTR-antibody pairs to activate Jurkat T cells was analyzed in a reporter assay in Jurkat cells reflective of Jurkat cell activation. Jurkat cells were transduced with lentivirus encoding firefly luciferase downstream of a minimal CMV promoter element and tandem repeats of the nuclear factor of activated T-cells (NFAT) consensus binding site. In this cell line, upregulation of NFAT transcription factors results in binding to the transcriptional response elements and subsequent expression of luciferase, which is monitored by measuring light produce following luciferase cleavage of its substrate luciferin.

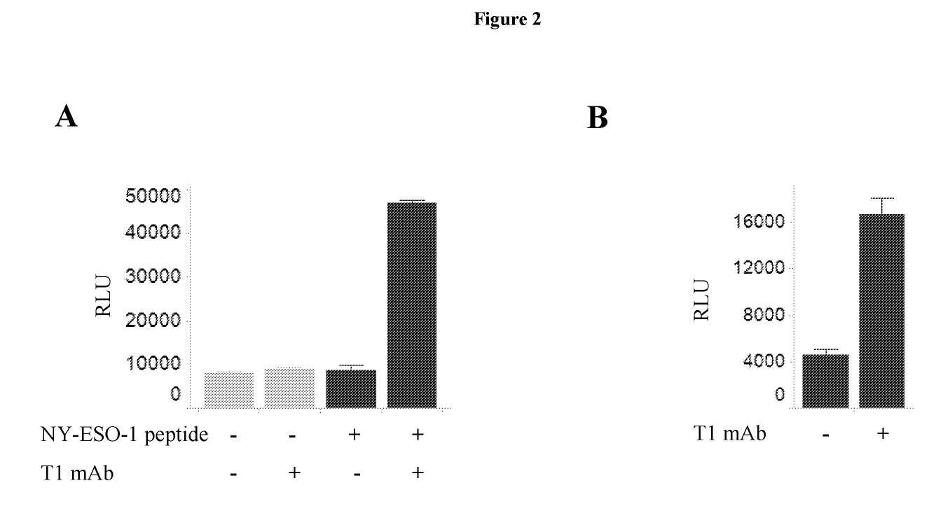

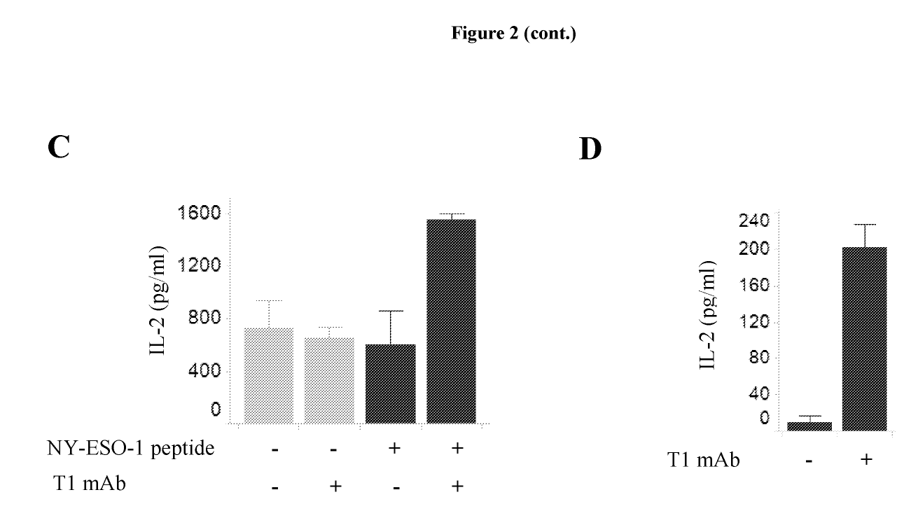

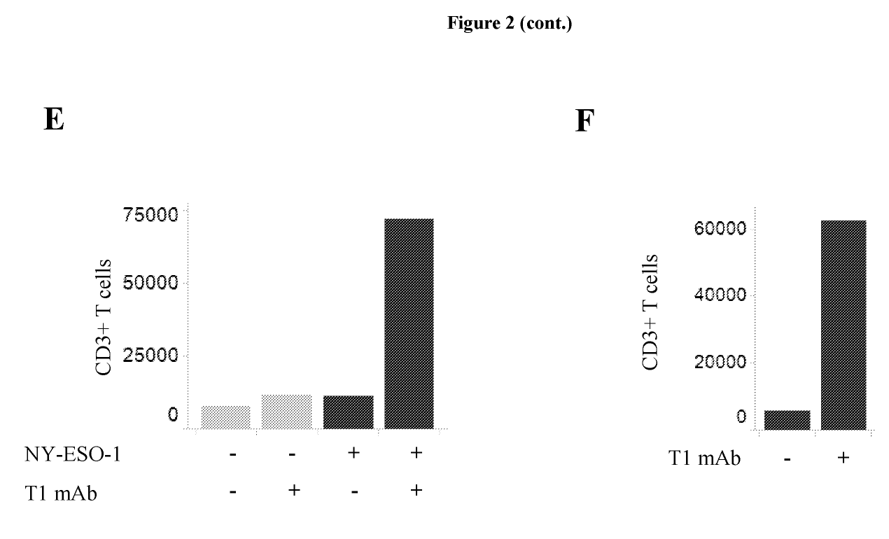

Jurkat cells with the NFAT reporter system (Jurkat-N) were transduced with gamma-retrovirus encoding ACTR construct SEQ ID NO: 10 to generate cells that stably expressed the ACTR variant. Jurkat-N cells expressing ACTR variant SEQ ID NO: 10 were mixed at a 1:1 ratio with target T2 cells that were labeled with and without NY-ESO-1 analog peptide (SLLMWITQV) (FIG. 2, panel A) or U266B1 tumor cells expressing endogenous target (FIG. 2, panel B) and afucosylated T1 mAb (anti-NY-ESO-1/HLA-A2) in a 30-μL reaction volume in RPMI-1640 media supplemented with 10% fetal bovine serum. Reactions were incubated for 5 hr in a CO₂ (5%) incubator at 37° C. Bright-Glo reagent (30 μL, Promega; Madison, Wis.) was added to lyse the cells and add the luciferin reagent. Reactions were incubated for 10 min in the dark and luminescence was measured using a Spectramax i3x system (Molecular Devices; Sunnyvale, Calif.) or an EnVision Multi-label plate reader (Perkin-Elmer; Waltham, Mass.).

The ability of afucosylated T1 mAb (anti-NY-ESO-1/HLA-A2) to activate ACTR variant SEQ ID NO: 10 Jurkat-N cells, as measured by an increase in luminescence, was evaluated. Afucosylated T1 mAb mediated ACTR activity in the presence of NY-ESO-1 peptide-pulsed T2 cells. ACTR activity was greater with T2 cells labeled with NY-ESO-1 peptide relative to the no peptide control (FIG. 2, panel A). The T1 antibody also mediated ACTR activation in response to endogenous target on U266B1 tumor cells (FIG. 2, panel B).

(iii) Primary T Cell Activity Assay

Gamma-retroviruses encoding ACTR variant SEQ ID NO: 149 were used to infect primary human T-cells to generate cells that express the ACTR variant on their cell surface. These cells were used in activation assays with target T2 cells that were labeled with and without NY-ESO-1 analog peptide (SLLMWITQV, SEQ ID NO: 101) (FIG. 2, panels C and E) or U266B1 tumor cells expressing endogenous target (FIG. 2, panels D and F) and the NY-ESO-1-targeting antibody T1.

T-cells (effector; E) and target cells (target; T) were incubated at a 1:1 effector-to-target ratio (50,000 target cells; 50,000 effector cells) in the presence of afucosylated anti-NY-ESO-1/HLA-A2 (T1) antibody in a 200-μL reaction volume in RPMI 1640 media supplemented with 10% fetal bovine serum. Reactions were incubated in a CO₂ (5%) incubator at 37° C. for 20-24 hr. After the incubation, 100 μL of the supernatant was collected. Supernatants were analyzed for IL-2 release by a time-resolved FRET assay.

T cell proliferation was evaluated after 6 days. Pelleted cells were washed with DPBS and stained with fixable viability dye eFluor450 (Affymetrix eBioscience) for 30 min. Cells were washed with MACS buffer (autoMACS buffer plus bovine serum albumin; Miltenyi) and then stained with anti-CD3 antibody CD3 Alexa Fluor 488 Clone OKT3 (BioLegend) for 30 min on ice. Cells were washed with MACS buffer and then analyzed by flow cytometry. Flow cytometry data was analyzed using the FlowJo software package.

Afucosylated T1 mAb mediated IL-2 release and T cell proliferation in the presence of NY-ESO-1 peptide-pulsed T2 cells. IL-2 release and T cell proliferation were greater with T2 cells labeled with NY-ESO-1 peptide relative to the no peptide control (FIG. 2, panels C and E). The T1 antibody also mediated IL-2 release and T cell proliferation in response to endogenous target on U266B1 tumor cells (FIG. 2, panels D and F).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a disease, the method comprising administering to a subject in need thereof
(i) a therapeutically effective amount of an immune cell that expresses a chimeric receptor comprising
(a) an Fc binding domain,
and
(b) a cytoplasmic signaling domain; and
(ii) a therapeutically effective amount of a bipartite targeting ligand, wherein the bipartite targeting ligand binds the chimeric receptor, and wherein the disease is an inflammatory disorder, an infectious disease, or cancer.

2. The method of claim 1, wherein the chimeric receptor further comprises one or more additional domains selected from the group consisting of:
a transmembrane domain;
a co-stimulatory signaling domain; and
a hinge domain.

3. The method of claim 1, wherein the chimeric receptor comprises, from N-terminus to C-terminus,
(a) the Fc binding domain,
(b) the transmembrane domain,
(c) the co-stimulatory domain, and
(d) the cytoplasmic signaling domain.

4. The method of claim 3, wherein the chimeric receptor further comprises a hinge domain, which is located between (a) and (b).

5. The method of claim 1, wherein the chimeric receptor further comprises a signal peptide.

6. The method of claim 1, wherein the Fc binding domain is an extracellular domain of an Fc receptor.

7. The method of claim 6, wherein the Fc receptor is a Fcγ receptor (FcγR).

8. The method of claim 7, wherein the FcγR is selected from the group consisting of CD16A, CD16B, CD64A, CD64B, CD64C, CD32A, and CD32B.

9. The method of claim 8, wherein the FcγR is CD16A.

10. The method of claim 6,
wherein the Fc binding domain is the extracellular domain of a wild-type Fc receptor and
wherein the bipartite targeting ligand comprises a wild-type Fc fragment.

11. The method of claim 6,
wherein the Fc binding domain is a mutated extracellular domain of an Fc receptor comprising a mutation at one or more residues involved in the Fc receptor/Fc interaction such that the mutated extracellular domain of the Fc receptor has altered binding activity to a wild-type Fc fragment; and
wherein the bipartite targeting ligand is afucosylated in the Fc fragment therein or comprises a mutation at one or more residues involved in the Fc receptor/Fc interaction such that the bipartite targeting ligand binds the mutated extracellular domain of the Fc receptor.

12. The method of claim 1, wherein the Fc binding domain is a single-chain antibody that binds to an IgG Fc.

13. The method of claim 12, wherein the Fc binding domain is a single-chain antibody that binds a wild-type Fc fragment.

14. The method of claim 12, wherein the Fc binding domain is a single-chain antibody that binds a mutated Fc fragment.

15. The method of claim 1, wherein the bipartite targeting ligand is an antibody that binds a T cell epitope complexed with a Major Histocompatibility Complex (MHC) molecule.

16. The method of claim 1, wherein the bipartite targeting ligand is an Fc-fusion protein comprising a variable region of a T cell receptor fused to an Fc region of an immunoglobulin G (IgG), and wherein the Fc-fusion protein binds to a T cell epitope complexed with a MHC molecule.

17. The method of claim 15, wherein the T cell epitope is a MHC Class I restricted T cell epitope.

18. The method of claim 15, wherein the T cell epitope is a MHC Class II restricted T cell epitope.

19. The method of claim 15, wherein the T cell epitope is derived from a cancer antigen.

20. The method of claim 19, wherein the cancer antigen is selected from the group consisting of WT1, HA-1H, NY-ESO-1, and HER-2.

21. The method of claim 19, wherein the T cell epitope has an amino acid sequence selected from the group consisting of SEQ ID NO: 75-77.

22. The method of claim 20, wherein the bipartite targeting ligand binds a peptide of SEQ ID NO: 75-77 complexed with HLA-A*02:01.

23. The method of claim 15, wherein the T cell epitope is derived from a viral antigen.

24. The method of claim 23, wherein the viral antigen is selected from the group consisting of HPV E6, HPV E7, EBV LMP2, and HIV gag.

25. The method of claim 23, wherein the T cell epitope has an amino acid sequence selected from the group consisting of SEQ ID NO: 91-96.

26. The method of claim 25,
wherein the bipartite targeting ligand binds to a peptide of SEQ ID NO: 95 complexed with HLA-B*57, or
wherein the bipartite targeting ligand binds to a peptide of SEQ ID NO: 96 complexed with HLA-B*2705.

27. The method of claim 15, wherein the T cell epitope is derived from an auto-antigen.

28. The method of claim 27, wherein the auto-antigen is a heat-shock protein.

29. The method of claim 27, wherein the T cell epitope has an amino acid sequence selected from the group consisting of SEQ ID NO: 97-100.

30. The method of claim 1, wherein the subject is a human patient having a cancer.

31. The method of claim 30, wherein the cancer is selected from the group consisting of carcinoma, lymphoma, sarcoma, blastoma, and leukemia.

32. The method of claim 1, wherein the subject is a human patient having an infectious disease.

33. The method of claim 32, wherein the infectious disease is a viral infection.

34. The method of claim 33, wherein the viral infection is an infection with Epstein-Barr Virus, Human Papillomavirus, or Human Immunodeficiency Virus.

35. The method of claim 1, wherein the subject is a human patient having an inflammatory disorder.

36. The method of claim 35, wherein the inflammatory disorder is acute inflammation or chronic inflammation.

37. The method of claim 35, wherein the inflammatory disorder is rheumatoid arthritis, multiple sclerosis, or systemic lupus erythematosus.

38. The method of claim 1, wherein the immune cell is a T lymphocyte or an NK cell.

39. The method of claim 38, wherein the T lymphocyte or NK cell is an autologous T lymphocyte or an autologous NK cell isolated from the subject.

40. The method of claim 38, wherein prior to the administration step, the autologous T lymphocyte or autologous NK cells are activated and/or expanded ex vivo.

41. The method of claim 38, wherein the T lymphocyte or NK cell is an allogeneic T lymphocyte or an allogeneic NK cell.

42. The method of claim 41, wherein the allogeneic T lymphocyte is engineered to reduce graft-versus-host effects or host-versus-graft effects.

43. The method of claim 42, wherein the endogenous T cell receptor of the allogeneic T lymphocyte has been inhibited or eliminated.

44. The method of claim 1, wherein the immune cell further expresses an exogenous polypeptide comprising a co-stimulatory domain or a ligand of a co-stimulatory factor.

45. The method of claim 1, wherein the immune cell is administered prior to, concurrently, or after the bipartite targeting ligand.

46. A kit for antibody-coupled T cell receptor immunotherapy, comprising:
(i) immune cells that express a chimeric receptor comprising
(a) an Fc binding domain,
and
(b) a cytoplasmic signaling domain; and
(ii) a bipartite targeting ligand that binds the chimeric receptor.

47. The kit of claim 46, wherein the chimeric receptor further comprises one or more additional domains selected from the group consisting of:
a transmembrane domain;
a co-stimulatory signaling domain; and
a hinge domain.

48. The kit of claim 46, wherein the chimeric receptor comprises, from N-terminus to C-terminus,
(a) the Fc binding domain,
(b) the transmembrane domain,
(c) the co-stimulatory domain, and
(d) the cytoplasmic signaling domain.

49. The kit of claim 48, wherein the chimeric receptor further comprises a hinge domain, which is located between (a) and (b).

50. The kit of claim 46, wherein the chimeric receptor further comprises a signal peptide.

51. The kit of claim 46, wherein the Fc binding domain is an extracellular domain of an Fc receptor.

52. The kit of claim 51, wherein the Fc receptor is a Fcγ receptor (FcγR).

53. The kit of claim 52, wherein the FcγR is selected from the group consisting of CD16A, CD16B, CD64A, CD64B, CD64C, CD32A, and CD32B.

54. The kit of claim 53, wherein the FcγR is CD16A.

55. The kit of claim 51,
wherein the Fc binding domain is the extracellular domain of a wild-type Fc receptor and
wherein the bipartite targeting ligand comprises a wild-type Fc fragment.

56. The kit of claim 51,
wherein the Fc binding domain is a mutated extracellular domain of an Fc receptor comprising a mutation at one or more residues involved in the Fc receptor/Fc interaction such that the mutated extracellular domain of the Fc receptor has altered binding activity to a wild-type Fc fragment; and
wherein the bipartite targeting ligand is afucosylated in the Fc fragment therein or comprises a mutation at one or more residues involved in the Fc receptor/Fc interaction such that the bipartite targeting ligand binds the mutated extracellular domain of the Fc receptor.

57. The kit of claim 46, wherein the Fc binding domain is a single-chain antibody that binds to an IgG Fc.

58. The kit of claim 57, wherein the Fc binding domain is a single-chain antibody that binds a wild-type Fc fragment.

59. The kit of claim 57, wherein the Fc binding domain is a single-chain antibody that binds a mutated Fc fragment.

60. The kit of claim 46, wherein the bipartite targeting ligand is an antibody that binds a T cell epitope complexed with a Major Histocompatibility Complex (MHC) molecule.

61. The kit of claim 46, wherein the bipartite targeting ligand is an Fc-fusion protein comprising a variable region of a T cell receptor fused to an Fc region of an immunoglobulin G (IgG), and wherein the Fc-fusion protein binds to a T cell epitope complexed with a MHC molecule.

62. The kit of claim 60, wherein the T cell epitope is a MHC Class I restricted T cell epitope.

63. The kit of claim 60, wherein the T cell epitope is a MHC Class II restricted T cell epitope.

64. The kit of claim 60, wherein the T cell epitope is derived from a cancer antigen.

65. The kit of claim 64, wherein the cancer antigen is selected from the group consisting of WT1, HA-1H, NY-ESO-1, and HER-2.

66. The kit of claim 64, wherein the T cell epitope has an amino acid sequence selected from the group consisting of SEQ ID NO: 75-77.

67. The kit of claim 65, wherein the bipartite targeting ligand binds a peptide of SEQ ID NO: 75-77 complexed with HLA-A*02:01.

68. The kit of claim 60, wherein the T cell epitope is derived from a viral antigen.

69. The kit of claim 68, wherein the viral antigen is selected from the group consisting of HPV E6, HPV E7, EBV LMP2, and HIV gag.

70. The kit of claim 68, wherein the T cell epitope has an amino acid sequence selected from the group consisting of SEQ ID NO: 91-96.

71. The kit of claim 70,
wherein the bipartite targeting ligand binds to a peptide of SEQ ID NO: 95 complexed with HLA-B*57, or
wherein the bipartite targeting ligand binds to a peptide of SEQ ID NO: 96 complexed with HLA-B*2705.

72. The kit of claim 60, wherein the T cell epitope is derived from an auto-antigen.

73. The kit of claim 72, wherein the auto-antigen is a heat-shock protein.

74. The kit of claim 72, wherein the T cell epitope has an amino acid sequence selected from the group consisting of SEQ ID NO: 97-100.

75. The kit of claim 46, wherein the immune cell is a T lymphocyte or an NK cell.

76. The kit of claim 75, wherein the T lymphocyte or NK cell is an autologous T lymphocyte or an autologous NK cell isolated from the subject.

77. The kit of claim 76, wherein prior to the administration step, the autologous T lymphocyte or autologous NK cells are activated and/or expanded ex vivo.

78. The kit of claim 75, wherein the T lymphocyte or NK cell is an allogeneic T lymphocyte or an allogeneic NK cell.

79. The kit of claim 78, wherein the allogeneic T lymphocyte is engineered to reduce graft-versus-host effects or host-versus-graft effects.

80. The kit of claim 79, wherein the endogenous T cell receptor of the allogeneic T lymphocyte has been inhibited or eliminated.

81. The kit of claim 46, wherein the immune cell further expresses an exogenous polypeptide comprising a co-stimulatory domain or a ligand of a co-stimulatory factor.


SEQUENCE LISTING (159 sequences)

SEQ ID NO: 1 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, Artificial Sequence
  Synthetic Polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, Artificial Sequence
  Synthetic Polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, Artificial Sequence
  Synthetic Polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic Polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 436 aa, Artificial Sequence; identical to SEQ ID NO: 1
  Synthetic Polypeptide

SEQ ID NO: 58 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHRVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHLVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLKGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVFSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVWSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFYHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFFHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFWHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKWFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 254 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 74 - protein, 238 aa, Artificial Sequence
  Synthetic Polypeptide
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 75 - protein, 9 aa, Artificial Sequence
  Synthetic Polypeptide
RMFPNAPYL

SEQ ID NO: 76 - protein, 9 aa, Artificial Sequence
  Synthetic Polypeptide
SLLMWITQC

SEQ ID NO: 77 - protein, 9 aa, Artificial Sequence
  Synthetic Polypeptide
VLHDDLLEA

SEQ ID NO: 78 - protein, 223 aa, Artificial Sequence
  Synthetic Polypeptide
TCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 223 aa, Artificial Sequence; identical to SEQ ID NO: 78
  Synthetic Polypeptide

SEQ ID NO: 80 - protein, 223 aa, Artificial Sequence
  Synthetic Polypeptide
TCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 223 aa, Artificial Sequence
  Synthetic Polypeptide
TCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 82 - protein, 300 aa, Artificial Sequence
  Synthetic Polypeptide
  MISC_FEATURE (16)..(300): May be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 83 - protein, 15 aa, Artificial Sequence
  Synthetic Polypeptide
GGGGSGGGGSGGGGS

SEQ ID NO: 84 - protein, 30 aa, Artificial Sequence
  Synthetic Polypeptide
GGGGSGGGGSGGGGSGGGGSGGGGSGGGGS

SEQ ID NO: 85 - protein, 45 aa, Artificial Sequence
  Synthetic Polypeptide
GGGGSGGGGSGGGGSGGGGSGGGGSGGGGSGGGGSGGGGSGGGGS

SEQ ID NO: 86 - protein, 60 aa, Artificial Sequence
  Synthetic Polypeptide
GGGGSGGGGSGGGGSGGGGSGGGGSGGGGSGGGGSGGGGSGGGGSGGGGSGGGGSGGGGS

SEQ ID NO: 87 - protein, 75 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 88 - protein, 150 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 89 - protein, 225 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 90 - protein, 300 aa, Artificial Sequence; identical to SEQ ID NO: 82
  Synthetic Polypeptide

SEQ ID NO: 91 - protein, 10 aa, Artificial Sequence
  Synthetic Polypeptide
YMLDLQPETT

SEQ ID NO: 92 - protein, 10 aa, Artificial Sequence
  Synthetic Polypeptide
TIHDIILECV

SEQ ID NO: 93 - protein, 9 aa, Artificial Sequence
  Synthetic Polypeptide
CLGGLLTMV

SEQ ID NO: 94 - protein, 9 aa, Artificial Sequence
  Synthetic Polypeptide
FLYALALLL

SEQ ID NO: 95 - protein, 10 aa, Artificial Sequence
  Synthetic Polypeptide
TSTLQEQIGW

SEQ ID NO: 96 - protein, 10 aa, Artificial Sequence
  Synthetic Polypeptide
KRWIILGLNK

SEQ ID NO: 97 - protein, 15 aa, Artificial Sequence
  Synthetic Polypeptide
VLRCINEPTAAALAY

SEQ ID NO: 98 - protein, 15 aa, Artificial Sequence
  Synthetic Polypeptide
VLRIINEPTAAAIAY

SEQ ID NO: 99 - protein, 15 aa, Artificial Sequence
  Synthetic Polypeptide
VMRIVNEPTAAAIAY

SEQ ID NO: 100 - protein, 15 aa, Artificial Sequence
  Synthetic Polypeptide
VLRIVNEPTAAALAY

SEQ ID NO: 101 - protein, 9 aa, Artificial Sequence
  Synthetic Polypeptide
SLLMWITQV

SEQ ID NO: 102 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKAVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSDNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSDQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSENVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSEQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSDNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSENVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSDQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSEQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHNVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHNVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHEVTYLQNGKGRKAFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHDVTYLQNGKGRKAFHHNSDFYIPKATLKDSGSYFCRGLWGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLNGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLQGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLWGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHLVTYLQNGKGRKYFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKWFHHNSDFYIPKATLKDSGSYFCRGLVGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGKLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGRLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGFLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGVLLLQAPRWVFKEEDPIHLRCHSWKNTALHKVTYLQNGKGRKYFHHNSDFYIPKATLKDSGSYFCRGLDGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGKLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGRLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGFLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGVLLLQAPRWVFKEEDPIHLRCHSWKNTALHKVTYLQNGKGRKYFHHNSDFYIPKATLKDSGSYFCRGLDGSKQVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKYFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKAFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHMVTYLQNGKGRKAFHHNSDFYIPKATLKDSGSYFCRGLWGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 393 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 428 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 431 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 483 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 469 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 472 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 390 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 397 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 390 aa, Artificial Sequence; identical to SEQ ID NO: 150
  Synthetic Polypeptide

SEQ ID NO: 153 - protein, 383 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 386 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 395 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 394 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 438 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 419 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 409 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR